(19) United States
(12) Patent Application Publication
(10) Pub. No.: US 2014/0316138 A1
(43) Pub. Date: Oct. 23, 2014

(54) PROCESS FOR PREPARING NEW TIOTROPIUM SALTS, NEW TIOTROPIUM SALTS AS SUCH AND PHARMACEUTICAL COMPOSITIONS THEREOF

(71) Applicants: Mihaela Maria POP, Amsterdam (NL); Stéphanie MULDER HOUDAYER, Maarssen (NL); Peter SIEGER, Mittelbiberach (DE); Michael P. PIEPER, Biberach an der Riss (DE)

(72) Inventors: Mihaela Maria POP, Amsterdam (NL); Stéphanie MULDER HOUDAYER, Maarssen (NL); Peter SIEGER, Mittelbiberach (DE); Michael P. PIEPER, Biberach an der Riss (DE)

(73) Assignee: Boehringer Ingelheim International GmbH, Ingelheim am Rhein (DE)

(21) Appl. No.: 14/323,361

(22) Filed: Jul. 3, 2014

Related U.S. Application Data
(62) Division of application No. 11/424,244, filed on Jun. 15, 2006, now abandoned.

(30) Foreign Application Priority Data
Jun. 15, 2005 (EP) 05105270

(51) Int. Cl. C07D 451/00

(52) U.S. Cl. CPC C07D 451/00
USPC 546/91

(57) ABSTRACT

The invention relates to a process for preparing new tiotropium salts, these new tiotropium salts as such, pharmaceutical formulations containing them and their use for preparing a medicament.

The invention relates to a process for preparing new tiotropium salts, these new tiotropium salts as such, pharmaceutical formulations containing them and their use for preparing a medicament.

### BACKGROUND OF THE INVENTION

Tiotropium bromide is known from European Patent Application EP 418 716 A1 and has the following chemical structure:

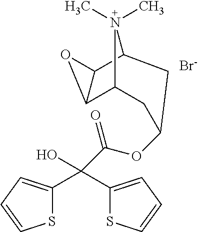

Tiotropium bromide is a highly effective anticholinergic with a long-lasting effect, which may be used to treat respiratory complaints, particularly COPD (chronic obstructive pulmonary disease) and asthma. By tiotropium is meant the free ammonium cation.

The aim of the present invention is to provide an alternative method of synthesis for preparing tiotropium salts which enables other tiotropium salts to be synthesised by a simple, non-aggressive method which is universally applicable. Another object of the invention is to provide new tiotropium salts as such which are characterized by advantageous physichochemical properties. Another object of the invention is to provide new tiotropium salts as such which are characterized by unexpected, new pharmacological properties.

### DETAILED DESCRIPTION OF THE INVENTION

The problem stated above is solved by the process according to the invention as described hereinafter.

The invention relates to a process for preparing new tiotropium salts of formula 1

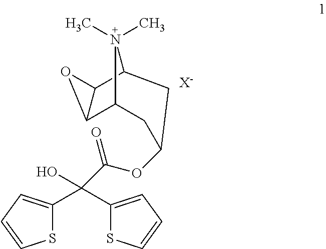

wherein

X⁻ denotes an anion which is different from HCO₃⁻ (=bicarbonate)

characterised in that tiotropium bicarbonate of formula 2

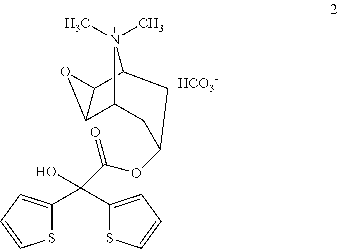

is reacted in a suitable solvent with an acid HX wherein X may have the meanings given above.

In a preferred embodiment the invention is directed to a process for the preparation of tiotropium salts of formula 1 wherein
X⁻ denotes halide, HSO₄⁻, H₂PO₄⁻ or an anion selected from optionally substituted alkylsulphonate, optionally substituted alkenylsulphonate, optionally substituted alkinylsulphonate, optionally substituted cycloalkylsulphonate, optionally substituted alkylsulphate, optionally substituted alkenylsulphate, optionally substituted alkinylsulphate, optionally substituted cycloalkylsulphate, optionally substituted arylsulphonate, optionally substituted arylsulphate, optionally substituted heterocyclylsulphonate and optionally substituted heterocyclylsulphate; or
X⁻ denotes R—COO⁻, wherein R is hydrogen, COOH, COO—C₁-C₆-alkyl or a group selected from optionally substituted alkyl, optionally substituted alkenyl, optionally substituted alkinyl, optionally substituted cycloalkyl, optionally substituted aryl, and optionally substituted heterocyclyl.

In a yet another preferred embodiment the invention is directed to a process for the preparation of tiotropium salts of formula 1 wherein
X⁻ denotes halide, HSO₄⁻, H₂PO₄⁻ or an anion selected from optionally substituted C₁-C₁₀-alkylsulphonate, optionally substituted C₂-C₁₀-alkenylsulphonate, optionally substituted C₂-C₁₀-alkinylsulphonate, optionally substituted C₃-C₈-cycloalkylsulphonate, optionally substituted C₁-C₁₀-alkylsulphate, optionally substituted C₂-C₁₀-alkenylsulphate, optionally substituted C₂-C₁₀-alkinylsulphate, optionally substituted C₃-C₈-cycloalkylsulphate, optionally substituted C₆-C₁₀-arylsulphonate, optionally substituted C₆-C₁₀-arylsulphate, optionally substituted heterocyclylsulphonate and optionally substituted heterocyclylsulphate; or
X⁻ denotes R—COO⁻, wherein R is hydrogen, COOH, COO—C₁-C₆-alkyl or a group selected from optionally substituted C₁-C₁₀-alkyl, optionally substituted C₂-C₁₀-alkenyl, optionally substituted C₂-C₁₀-alkinyl, optionally substituted C₃-C₈-cycloalkyl, optionally substituted C₆-C₁₀-aryl, and optionally substituted heterocyclyl.

In a yet another preferred embodiment the invention is directed to a process for the preparation of tiotropium salts of formula 1 wherein
X⁻ denotes halide, HSO₄⁻, H₂PO₄⁻ or an anion selected from C₁-C₁₀-alkylsulphonate, C₂-C₁₀-alkenylsulphonate, C₂-C₁₀-alkinylsulphonate, C₃-C₈-cycloalkylsulphonate, C₁-C₁₀-alkylsulphate, C₂-C₁₀-alkenylsulphate, C₂-C₁₀-alkinylsulphate, C₃-C₈-cycloalkylsulphate, C₆-C₁₀-arylsulphonate, C₆-C₁₀-arylsulphate, heterocyclylsulphonate and heterocyclylsulphate, which is optionally substituted by one or more non-interfering groups; or
X⁻ denotes R—COO⁻, wherein R is hydrogen, COOH, COO—C₁-C₆-alkyl or a group selected from C₁-C₁₀-alkyl, C₂-C₁₀-alkenyl, C₂-C₁₀-alkinyl, C₃-C₈-cycloalkyl, C₆-C₁₀-aryl, and heterocyclyl, which is optionally substituted by one or more non-interfering groups.

In a yet another preferred embodiment the invention is directed to a process for the preparation of tiotropium salts of formula 1 wherein
X⁻ denotes halide, HSO₄⁻, H₂PO₄⁻ or an anion selected from C₁-C₁₀-alkylsulphonate, C₂-C₁₀-alkenylsulphonate, C₂-C₁₀-alkinylsulphonate, C₃-C₈-cycloalkylsulphonate, C₁-C₁₀-alkylsulphate, C₂-C₁₀-alkenylsulphate, C₂-C₁₀-alkinylsulphate, C₃-C₈-cycloalkylsulphate, C₆-C₁₀-arylsulphonate, C₆-C₁₀-arylsulphate, heterocyclylsulphonate and heterocyclylsulphate, which is optionally substituted by one or more non-interfering groups which are preferably selected from halogen, OH, ═O, CN, NO₂, NH₂, COOH, COO—C₁-C₆-alkyl, C₁-C₆-alkyl, C₁-C₆-haloalkyl, C₁-C₆-alkyloxy, C₃-C₈-cycloalkyl, C₆-C₁₀-aryl; or
X⁻ denotes R—COO⁻, wherein R is hydrogen, COOH, COO—C₁-C₆-alkyl or a group selected from C₁-C₁₀-alkyl, C₂-C₁₀-alkenyl, C₂-C₁₀-alkinyl, C₃-C₈-cycloalkyl, C₆-C₁₀-aryl, and heterocyclyl, which is optionally substituted by one or more non-interfering groups which are preferably selected from halogen, OH, ═O, CN, NO₂, NH₂, COOH, COO—C₁-C₆-alkyl, C₁-C₆-alkyl, C₁-C₆-haloalkyl, C₁-C₆-alkyloxy, C₃-C₈-cycloalkyl, C₆-C₁₀-aryl.

In a yet another preferred embodiment the invention is directed to a process for the preparation of tiotropium salts of formula 1 wherein
X⁻ denotes halide, HSO₄⁻, H₂PO₄⁻ or an anion selected from C₁-C₆-alkylsulphonate, C₂-C₆-alkenylsulphonate, C₂-C₆-alkinylsulphonate, C₃-C₆-cycloalkylsulphonate, C₁-C₆-alkylsulphate, C₂-C₆-alkenylsulphate, C₂-C₆-alkinylsulphate, C₃-C₆-cycloalkylsulphate, C₆-C₁₀-arylsulphonate, C₆-C₁₀-arylsulphate, heterocyclylsulphonate and heterocyclylsulphate, which is optionally substituted by one or more non-interfering groups being preferably selected from halogen, OH, ═O, CN, NO₂, NH₂, COOH, COO—C₁-C₄-alkyl, C₁-C₄-alkyl, C₁-C₄-haloalkyl, C₁-C₄-alkyloxy, C₃-C₆-cycloalkyl, C₆-C₁₀-aryl; or
X⁻ denotes R—COO⁻, wherein R is hydrogen, COOH, COO—C₁-C₆-alkyl or a group selected from C₁-C₆-alkyl, C₂-C₆-alkenyl, C₂-C₆-alkinyl, C₃-C₆-cycloalkyl, C₆-C₁₀-aryl, and heterocyclyl, which is optionally substituted by one or more non-interfering groups being preferably selected from halogen, OH, ═O, CN, NO₂, NH₂, COOH, COO—C₁-C₆-alkyl, C₁-C₆-alkyl, C₁-C₆-haloalkyl, C₁-C₆-alkyloxy, C₃-C₈-cycloalkyl, C₆-C₁₀-aryl.

In a yet another preferred embodiment the invention is directed to a process for the preparation of tiotropium salts of formula 1 wherein
X⁻ denotes chloride, bromide, iodide, HSO₄⁻, H₂PO₄⁻ or an anion selected from C₁-C₆-alkylsulphonate, C₁-C₆-alkylsulphate, phenylsulphonate, and naphthylsulphonate, which is optionally substituted by one or more non-interfering groups being preferably selected from chlorine, bromine, fluorine, OH, ═O, CN, NO₂, NH₂, COOH, COO—C₁-C₄-alkyl, C₁-C₄-alkyl, C₁-C₄-fluoroalkyl, C₁-C₄-alkyloxy, C₃-C₆-cycloalkyl, phenyl, and naphthyl; or
X⁻ denotes R—COO⁻, wherein R is hydrogen, COOH, or a group selected from C₁-C₆-alkyl, C₂-C₆-alkenyl, C₂-C₆-alkinyl, C₃-C₆-cycloalkyl, phenyl, naphthyl, and heterocyclyl, which is optionally substituted by one or more non-interfering groups being preferably selected from chlorine, bromine, fluorine, OH, ═O, CN, NO₂, NH₂, COOH, COO—C₁-C₄-alkyl, C₁-C₄-alkyl, C₁-C₄-fluoroalkyl, C₁-C₄-alkyloxy, C₃-C₆-cycloalkyl, phenyl, naphthyl.

In a yet another preferred embodiment the invention is directed to a process for the preparation of tiotropium salts of formula 1 wherein
X⁻ denotes chloride, bromide, iodide, HSO₄⁻, H₂PO₄⁻ or an anion selected from methanesulphonate, benzenesulphonate, toluenesulphonate, trifluoromethansulphonate, or
X⁻ denotes R—COO⁻, wherein R is hydrogen, COOH, methyl, ethyl, propyl, —CH₂—OH, —CH₂—CH₂—OH, —CH₂—CH₂—CH₂—OH, —CH₂—CH₂—CH₂—CH₂—OH, —C(OH)H—CH₃, —C(OH)H—CH₂—CH₃, —C(OH)H—CH₂—CH₂—CH₃, —C(OH)H—CH₂—CH₂—CH₂—CH₃, —CH₂—COOH, —CH₂—CH₂—COOH, —CH₂—CH₂—CH₂—COOH, —CH₂—CH₂—CH₂—CH₂—COOH, —C(OH)H—COOH, —C(OH)H—CH₂—COOH, —C(OH)H—CH₂—CH₂—COOH, —C(OH)H—CH₂—CH₂—CH₂—COOH, —C(NH₂)H—COOH, —C(NH₂)H—CH₂—COOH, —C(NH₂)H—CH₂—CH₂—COOH, —C(NH₂)H—CH₂—CH₂—CH₂—COOH, —C(OH)H—C(OH)H—COOH, —C(OH)H—C(OH)H—CH₂—COOH, —C(OH)H—C(OH)H—CH₂—CH₂—COOH, —C(OH)H—C(OH)H—C(OH)H—COOH, —C(OH)H—C(OH)H—C(OH)H—C(OH)H—COOH, —CH═CH2, —CH═CH—CH₃, —CH═CH—COOH, or
X⁻ denotes R—COO⁻, wherein R is a group selected from cyclopentyl and cyclohexyl, being optionally substituted by one or more groups selected from among ═O, OH, COOH, methyl, ethyl and fluorine, or
X⁻ denotes R—COO⁻, wherein R is a group selected from phenyl, benzyl, phenylethyl, and naphthyl, being optionally substituted by one or more groups selected from among ═O, OH, COOH, methyl, ethyl, fluorine, chlorine, CF₃, phenyl, benzyl, naphthyl, napthylmethyl, and naphthylmethyl which is substituted by OH and/or COOH.

In a yet another preferred embodiment the invention is directed to a process for the preparation of tiotropium salts of formula 1 wherein
X⁻ denotes chloride, iodide, HSO₄⁻, H₂PO₄⁻, benzenesulphonate, toluenesulphonate, methanesulphonate, trifluoromethanesulphonate, xinafoate, malate, aspartate, succinate, camphorate, acetate, propionate, fumarate, isobutyrate, malonate, oxalate, salicylate, tartrate, ethandisulfonate, gluconate, glutarate, lactate, citrate, maleinate, saccharinate and pamoate.

In case X⁻ denotes an anion which possesses a second acidic hydrogen the process according to the invention may also lead to compounds according to formula 1a

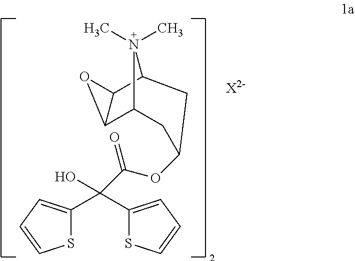

wherein X²⁻ is a di-anion selected from those anions X⁻ mentioned hereinbefore that are able to form a di-anion by donation of another H⁺. For a person of ordinary skill in the art it is clear which of the aforementioned groups X are capable to form such a di-anion. As an example are to be mentioned for instance HSO₄⁻, H₂PO₄⁻, and all those groups X that possess a second COOH-group as specified hereinbefore.

The invention relates to a process for the preparation of compounds of formula 1 optionally in the form of the individual optical isomers, mixtures of the individual enantiomers or racemates, and optionally to the hydrates and/or solvates thereof.

The alkyl groups meant here (including those which are components of other groups) are branched and unbranched alkyl groups having 1 to 10 carbon atoms, preferably 1 to 4 carbon atoms, such as: methyl, ethyl, n-propyl, iso-propyl, butyl, iso-butyl, sec.-butyl, tert.-butyl, pentyl, iso-pentyl, hexyl, heptyl and octyl.

Unless otherwise specified, substituted alkyl groups (including those which are components of other groups) may, for example, carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

Alkenyl groups (including those which are components of other groups) are the branched and unbranched alkenyl groups with 2 to 10 carbon atoms, preferably 2 to 3 carbon atoms, provided that they have at least one double bond, e.g. the alkyl groups mentioned above provided that they have at least one double bond, such as for example vinyl (provided that no unstable enamines or enolethers are formed), propenyl, iso-propenyl, butenyl, pentenyl and hexenyl.

Unless otherwise specified, substituted alkenyl groups, (including those which are components of other groups), may for example carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

The term alkinyl groups (including those which are components of other groups) refers to alkinyl groups having 2 to 10 carbon atoms provided that they have at least one triple bond, e.g. ethinyl, propargyl, butinyl, pentinyl and hexinyl.

Unless otherwise specified, substituted alkinyl groups, (including those which are components of other groups), may for example carry one or more of the following substituents: halogen, hydroxy, mercapto, C₁₋₆-alkyloxy, amino, alkylamino, dialkylamino, cyano, nitro, ═O, —CHO, —COOH, —COO—C₁₋₆-alkyl, —S—C₁₋₆-alkyl.

Examples of cycloalkyl groups having 3 to 8 carbon atoms include cyclopropyl, cyclobutyl, cyclopentyl, cyclohexyl, cycloheptenyl and cyclooctyl which may also be substituted by branched or unbranched C₁₋₄-alkyl, hydroxy and/or halogen or as hereinbefore defined. The term halogen generally refers to fluorine, chlorine, bromine or iodine.

The word aryl denotes an aromatic ring system having 6 to 10 carbon atoms which, unless otherwise specified, may carry one or more of the following substituents, for example: C₁₋₆-alkyl, C₁₋₆-alkyloxy, halogen, hydroxy, mercapto, amino, alkylamino, dialkylamino, CF₃, cyano, nitro, —CHO, —COOH, —COO—C₁₋₆-alkyl, —S—C₁₋₆-alkyl. The preferred aryl group is phenyl.

The term heterocyclyl denotes cyclic groups possessing at least one heteroatom which may contain nitrogen, oxygen or sulphur as heteroatoms. Examples include furan, tetrahydrofuran, tetrahydrofuranone, γ-butyrolactone, α-pyran, γ-pyran, dioxolane, tetrahydropyran, dioxane, thiophene, dihydrothiophene, thiolane, dithiolane, pyrrole, pyrroline, pyrrolidine, pyrazole, pyrazoline, imidazole, imidazoline, imidazolidine, triazole, tetrazole, pyridine, piperidine, pyridazine, pyrimidine, pyrazine, piperazine, triazine, tetrazine, morpholine, thiomorpholine, oxazole, isoxazole, oxazine, thiazole, isothiazole, thiadiazole, oxadiazole and pyrazolidine, preferably morpholine, piperazine and piperidine, wherein the above-mentioned heterocycles may also be substituted by benzyl or C₁₋₄-alkyl, preferably methyl.

“═O” means an oxygen atom linked by a double bond.

A non-interfering group is to be understood as a group which leaves the activity of the compounds 1 in the intended use qualitatively intact. The intended use of the compounds of formula 1 is specified in more detail below. Examples of non-interfering groups within the scope of the instant invention are selected from halogen, OH, ═O, CN, NO₂, NH₂, COOH, COO—C₁-C₆-alkyl, C₁-C₆-alkyl, C₁-C₆-haloalkyl, C₁-C₆-alkyloxy, C₃-C₈-cycloalkyl, C₆-C₁₀-aryl. Further examples of non-interfering groups include phenyl-C₁-C₆-alkylen or napthyl-C₁-C₆-alkylene which are optionally substituted by one or more groups selected from methyl, ethyl, OH, COOH, COO—C₁-C₄-alkyl and CF₃, preferably OH or COOH.

In the process according to the invention the tiotropium bicarbonate is dissolved or suspended in a suitable solvent. Suitable solvents include water or organic solvents, preferably water or polar organic solvents that are suitable to dissolve tiotropium bicarbonate 2. Preferred solvents are protic solvents such as alcohols (for example methanol, ethanol, isopropanol) and water, preferably water of pH 2-6 as well as polar organic solvents selected from the group consisting of alcohols such as for example ethyleneglycol and diethyleneglycol, amides such as for example dimethylformamide and N-methyl-pyrrolidinone, ethers such as for example tetrahydrofuran, dioxane, dimethylether and nitriles such as for example acetonitrile. It is particularly preferable to use water, methanol, ethanol, isopropanol, ethyleneglycol, diethyleneglycol, dimethylformamide, N-methyl-pyrrolidinone, tetrahydrofuran, dioxane, dimethylether or acetonitrile as solvent, while water, particularly aqueous solutions with a pH of about 2-6 are particularly preferred according to the invention. The solution of tiotropium bicarbonate 2 in one of the aforementioned solvents is treated with the acid HX, wherein X may have the meanings specified herein. Preferably the tiotropium bicarbonate 2 solution is treated with HX at a pH below 5, preferably below 4, more preferably in a range of pH 2-3. This pH is adjusted by the acid HX.

Progress of the transformation can be detected for instance via ¹H-NMR or other methods, well known to the person of ordinary skill in the art.

When the transformation is complete, the solution is cooled down to a temperature below 15° C., preferably below 10° C., more preferably to 3-5° C. Usually, the salts of formula 1 crystallize from the reaction solution.

In case crystallization does not occur, the solvent is removed and the remaining residue, if not crystalline recrystallized from alcohol, preferably ethanol.

In a particular preferred embodiment according to the invention, the process is conducted using a suitable ion exchange resin. Preferred ion exchange resins are basic anion exchange resins based on polystyrene, optionally cross linked with divinyl benzene or polyethylenglycol. Of particular interest are ion exchange resins based on styrene-divenylbenzen basis. Within the scope of the invention the term ion exchange resin is occasionally also abbreviated by the term IER.

In a particular preferred embodiment of the invention an ion exchange resin as specified hereinbefore is already used for the preparation of the tiotropium bicarbonate 2 starting material.

This preparation of the bicarbonate 2 is preferably conducted as follows. An ion exchange resin (IER) suitably charged with bicarbonate-ions is treated with a solution of a tiotropium salt already known in the art in a suitable solvent. Preferably tiotropium bromide is used as the starting material, more preferably tiotropium bromide monohydrate as known from WO 02/30928 is used. Other tiotropium salts and salt forms that are suitable as staring materials for the preparation of the bicarbonate 2 are disclosed for instance in EP 0418716, WO 03/000265, WO 05/042526, WO 05/042528, and WO 05/042527. Suitable solvents include water or organic solvents, preferably water or polar organic solvents that are suitable to dissolve tiotropium bicarbonate 2. Preferred solvents are protic solvents such as alcohols (for example methanol, ethanol, isopropanol) and water, preferably water of pH 2-6 as well as polar organic solvents selected from the group consisting of alcohols such as for example ethyleneglycol and diethyleneglycol, amides such as for example dimethylformamide and N-methyl-pyrrolidinone, ethers such as for example tetrahydrofuran, dioxane, dimethylether and nitriles such as for example acetonitrile. It is particularly preferable to use water, methanol, ethanol, isopropanol, ethyleneglycol, diethyleneglycol, dimethylformamide, N-methyl-pyrrolidinone, tetrahydrofuran, dioxane, dimethylether or acetonitrile as solvent, while water, particularly aqueous solutions with a pH of about 2-6 are particularly preferred according to the invention.

The tiotropium bicarbonate 2 can be obtained from the IER in form of a solution by washing thereof with one of the solvents mentioned hereinbefore. Preferred solvent is water. The bicarbonate 2 containing solution can be detected in the washing procedure via a UV reader working at 240 nm and is preferably stored in form of this solution without further isolation of pure 2. 2 could be isolated via removal of the solvent according to known methods (i.e. evaporation of solvent or freeze drying). However, in these cases at least partial decomposition of the bicarbonate 2 may occur.

A yet another preferred embodiment according to the invention relates to the preparation of tiotropium bicarbonate 2 as specified hereinbefore.

The tiotropium bicarbonate 2 is an extremely valuable intermediate for the easy preparation of tiotropium salts 1. Consequently, in another embodiment, the instant invention relates to tiotropium bicarbonate 2

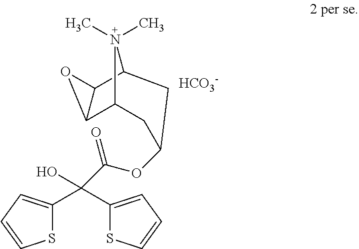

In another embodiment, the invention relates to solutions containing 2 dissolved or suspended, preferably dissolved in a solvent. In a yet another preferred embodiment the invention relates to solutions of 2 in a solvent such as alcohols (for example methanol, ethanol, isopropanol) and water, preferably water of pH 2-6 as well as solutions in polar organic solvents selected from the group consisting of alcohols such as for example ethyleneglycol and diethyleneglycol, amides such as for example dimethylformamide and N-methyl-pyrrolidinone, ethers such as for example tetrahydrofuran, dioxane, dimethylether and nitriles such as for example acetonitrile. Particularly preferred are solutions of 2 in a solvent selected from among water, methanol, ethanol, isopropanol, ethyleneglycol, diethyleneglycol, dimethylformamide, N-methyl-pyrrolidinone, tetrahydrofuran, dioxane, dimethylether or acetonitrile, while solutions in water, particularly with a pH of about 2-6 are particularly preferred according to the invention.

Another embodiment according to the invention is directed to the use of bicarbonate 2 as a starting material for the preparation of tiotropium salts 1. Another aspect of the invention is directed to new, crystalline salts of formula 1 per se, wherein X⁻ has the meanings defined hereinbefore.

In particular the invention relates to specific crystalline forms of tiotropium salts of formula 1, which are discussed in detail below.

In one preferred embodiment the invention relates to crystalline tiotropium benzenesulphonate, in particular to crystalline anhydrous tiotropium benzenesulphonate, preferably crystalline anhydrous tiotropium benzenesulphonate which is characterized by an orthorhombic elementary cell. In yet another preferred embodiment the invention relates to crystalline tiotropium benzenesulphonate which is characterized by an orthorhombic elementary cell with the parameters a=10.6460(7)Å, b=12.8410(10)Å, c=36.605(3)Å, and cell volume=5004.1(6) Å³ determined by single crystal X-ray structural analysis.

In another aspect the present invention relates to a method of preparing the new crystalline tiotropium benzenesulphonate which is explained by way of example in the experimental section that follows.

In another preferred embodiment the invention relates to crystalline tiotropium trifluoromethanesulphonate, in particular to crystalline anhydrous tiotropium trifluoromethanesulphonate, preferably crystalline anhydrous tiotropium trifluoromethanesulphonate which is characterized by a monoclinic elementary cell. In yet another preferred embodiment the invention relates to crystalline anhydrous tiotropium trifluoromethanesulphonate which is characterized by a monoclinic elementary cell with the parameters a=12.4500(7)Å, b=13.1090(9)Å, c=17.9840(14) Å, β=129.176(2)°, and cell volume=2275.3(3) Å³ determined by single crystal X-ray structural analysis.

In another aspect the present invention relates to a method of preparing the new crystalline tiotropium trifluoromethanesulphonate which is explained by way of example in the experimental section that follows.

In another preferred embodiment the invention relates to crystalline tiotropium salicylate, in particular to crystalline tiotropium salicylate monohydrate, preferably crystalline tiotropium salicylate monohydrate which is characterized by a triclinic elementary cell. In yet another preferred embodiment the invention relates to crystalline tiotropium salicylate monohydrate which is characterized by a triclinic elementary cell with the parameters a=10.8380(3)Å, b=10.8610(3)Å, c=12.2310(4)Å, a=76.199(2)°, β=71.878(2)°, γ=74.220(2)°, and cell volume=1297.95(7) Å³ determined by single crystal X-ray structural analysis. This form is also referred to as form I.

In another preferred embodiment the invention relates to crystalline tiotropium salicylate which is characterized by a X-ray powder diagram with the characteristic values d=13.50 Å; 7.23 Å; 5.46 Å; 4.30 Å; inter alia. This salicylate form is optionally also referred to as salicylate form II. In yet another preferred embodiment the invention relates to crystalline tiotropium salicylate form II which is characterized by a monoclinic elementary cell with the parameters a=13.273(2)Å, b=13.865(2)Å, c=28.042(4)Å, 13=101.98(2)°, and cell volume=5048.1(10) Å³ determined by single crystal X-ray structural analysis.

In yet another preferred embodiment the invention relates to crystalline tiotropium salicylate which is characterized by a X-ray powder diagram with the characteristic values d=10.80 Å; 6.49 Å; 5.42 Å; 4.28 Å; 3.93 Å; inter alia. This salicylate form is optionally also referred to as salicylate form III.

In yet another preferred embodiment the invention relates to crystalline tiotropium salicylate monohydrate which is characterized by a X-ray powder diagram with the characteristic values d=10.12 Å; 5.06 Å; 4.91 Å; 4.07 Å; 3.92 Å; inter alia. This salicylate monohydrate form is optionally also referred to as salicylate form IV.

In another aspect the present invention relates to methods of preparing the new crystalline tiotropium salicylate forms which are explained by way of example in the experimental section that follows.

In another preferred embodiment the invention relates to crystalline tiotropium hydrogenesulphate (X⁻=HSO₄), in particular to crystalline tiotropium hydrogenesulphate monohydrate, preferably crystalline tiotropium hydrogenesulphate monohydrate which is characterized by a triclinic elementary cell. In yet another preferred embodiment the invention relates to crystalline tiotropium hydrogenesulphate monohydrate which is characterized by a triclinic elementary cell with the parameters a=9.3750(2)Å, b=11.6470(2)Å, c=20.5450(5)Å, α=91.6260(9)°, β=95.7210(9)°, γ=91.8520(12)°, and cell volume=2229.85(8) Å³ determined by single crystal X-ray structural analysis. This hydrogensulphate form is optionally also referred to as hydrogensulphate form I.

In another preferred embodiment the invention relates to crystalline anhydrous tiotropium hydrogensulphate, which is characterized by a monoclinic elementary cell with the parameters a=8.0390(2)Å, b=15.989(1)Å, c=33.190(2)Å, β=90.265(2)°, and cell volume=4266.0(2) Å³ determined by single crystal X-ray structural analysis. This form is furthermore characterized by a X-ray powder diagram with the characteristic values d=5.67 Å; 4.79 Å; 4.65 Å; 3.95 Å; inter alia. This hydrogensulphate form is optionally also referred to as hydrogensulphate form II.

In yet another preferred embodiment the invention relates to crystalline anhydrous tiotropium hydrogensulphate which is characterized by a X-ray powder diagram with the characteristic values d=16.29 Å; 5.71 Å; 5.55 Å; 5.23 Å; 4.85 Å; 4.48 Å; 4.35 Å; 3.89 Å; inter alia. This hydrogensulphate form is optionally also referred to as hydrogensulphate form III.

In another aspect the present invention relates to methods of preparing the new crystalline tiotropium hydrogenesulphate forms which are explained by way of example in the experimental section that follows.

In another preferred embodiment the invention relates to crystalline tiotropium dihydrogenephosphate (X⁻=H₂PO₄⁻), in particular to crystalline tiotropium dihydrogenephosphate monohydrate, preferably crystalline tiotropium dihydrogenephosphate monohydrate which is characterized by a monoclinic elementary cell. In yet another preferred embodiment the invention relates to crystalline tiotropium dihydrogenephosphate monohydrate which is characterized by a monoclinic elementary cell with the parameters a=22.6740(17)Å, b=6.6690(9)Å, c=15.061(3)Å, β=96.476(8)°, and cell volume=2262.9(6) Å³ determined by single crystal X-ray structural analysis. This form is furthermore characterized by a X-ray powder diagram with the characteristic values d=7.35 Å; 6.26 Å; 4.90 Å; 4.22 Å; 3.63 Å; A; inter alia. In another aspect the present invention relates to a method of preparing the new crystalline tiotropium dihydrogenephosphate which is explained by way of example in the experimental section that follows.

In another preferred embodiment the invention relates to crystalline ditiotropium ethandisulphonate (tiotropium: ethandisulphonate=2:1), in particular to crystalline ditiotropium ethandisulphonate hydrate, preferably crystalline ditiotropium ethandisulphonate hydrate which is characterized by a triclinic elementary cell. In yet another preferred embodiment the invention relates to crystalline ditiotropium ethandisulphonate hydrate which is characterized by a triclinic elementary cell with the parameters a=9.2700(8)Å, b=12.8920(3)Å, c=22.579(2)Å, a=103.876(3)°, β=93.620(4)°, γ=90.327(5)°, and cell volume=2613.8(4) Å³ determined by single crystal X-ray structural analysis. In another aspect the present invention relates to a method of preparing the new crystalline ditiotropium ethandisulphonate which is explained by way of example in the experimental section that follows.

In another preferred embodiment the invention relates to crystalline tiotropium xinafoate, in particular to crystalline tiotropium xinafoate monohydrate, preferably crystalline tiotropium xinafoate monohydrate which is characterized by a monoclinic elementary cell. In yet another preferred embodiment the invention relates to crystalline tiotropium xinafoate monohydrate which is characterized by a monoclinic elementary cell with the parameters a=13.5460(16)Å, b=16.491(3)Å, c=13.263(2)Å, β=100.51(2)°, and cell volume=2913.0(7) Å³ determined by single crystal X-ray structural analysis. This form is furthermore characterized by a X-ray powder diagram with the characteristic values d=6.31 Å; 5.94 Å; 4.12 Å; inter alia. This xinafoate form is optionally also referred to as xinafoate form I.

In another preferred embodiment the invention relates to crystalline tiotropium xinafoate which is characterized by a monoclinic elementary cell with the parameters a=15. 9650(4)Å, b=13.2330(3)Å, c=14.1810(5)Å, β=111.781(2)°, and cell volume=2782.06(14) Å³ determined by single crystal X-ray structural analysis. This form is furthermore characterized by a X-ray powder diagram with the characteristic values d=14.57 Å; 6.55 Å; 5.99 Å; 4.91 Å; 3.92 Å; A; inter alia. This xinafoate form is optionally also referred to as xinafoate form II.

In yet another preferred embodiment the invention relates to crystalline tiotropium xinafoate monohydrate which is characterized by a monoclinic elementary cell with the parameters a=13.2470(6)Å, b=11.3590(6)Å, c=20.9500(7)Å, β=118.229(4)°, and cell volume=2777.5(2) Å³ determined by single crystal X-ray structural analysis. This form is furthermore characterized by a X-ray powder diagram with the characteristic values d=9.90 Å; 5.20 Å; 4.54 Å; 4.42 Å; A; inter alia. This xinafoate form is optionally also referred to as xinafoate form III.

In another aspect the present invention relates to methods of preparing the new crystalline tiotropium xinafoate forms which are explained by way of example in the experimental section that follows.

In another preferred embodiment the invention relates to crystalline tiotropium fumarate, in particular to crystalline tiotropium fumarate ethanolate, preferably crystalline tiotropium fumarate ethanolate which is characterized by an orthorhombic elementary cell. In yet another preferred embodiment the invention relates to crystalline tiotropium fumarate ethanolate which is characterized by an orthorhombic elementary cell with the parameters a=15.3830(7)Å, b=16.8490(7)Å, c=20.0900(12)Å, and cell volume=5207.1(4) Å³ determined by single crystal X-ray structural analysis. This fumarate form is optionally also referred to as fumarate form I.

In another preferred embodiment the invention relates to crystalline anhydrous tiotropium fumarate, in particular to crystalline anhydrous tiotropium fumarate which is characterized by a triclinic elementary cell. In yet another preferred embodiment the invention relates to crystalline anhydrous tiotropium fumarate which is characterized by an triclinic elementary cell with the parameters a=7.4980(3)Å, b=9.4900(4)Å, c=17.0110(7)Å, a=102.125(2)°, 13=96.182(2)°, γ=99.289(2)°, and cell volume=1155.27(8) Å³ determined by single crystal X-ray structural analysis. This form is furthermore characterized by a X-ray powder diagram with the characteristic values d=6.26 Å; 4.70 Å; 4.08 Å; inter alia. This fumarate form is optionally also referred to as fumarate form II.

In another preferred embodiment the invention relates to crystalline tiotropium fumarate which is characterized by a X-ray powder diagram with the characteristic values d=7.12 Å; 6.29 Å; 4.94 Å; 4.71 Å; 4.54 Å; 4.10 Å; 3.58 Å; inter alia. This fumarate form is optionally also referred to as fumarate form III.

In yet another preferred embodiment the invention relates to crystalline tiotropium fumarate, in which the ratio of tiotropium cation to fumarate counterion is 2:1. In particular the invention relates to such a tiotropium fumarate which is characterized by a monoclinic elementary cell with the parameters a=9.6910(2)Å, b=14.5710(4)Å, c=18.1580(4)Å, β=116.781(2)°, and cell volume=2289.01(9) Å³ determined by single crystal X-ray structural analysis. This form is furthermore characterized by a X-ray powder diagram with the characteristic values d=7.96 Å; 6.61 Å; 4.80 Å; 4.64 Å; 4.14 Å; 4.05 Å; 3.96 Å; inter alia. This fumarate form is optionally also referred to as fumarate form IV.

In another aspect the present invention relates to methods of preparing the new crystalline tiotropium fumarate forms which are explained by way of example in the experimental section that follows.

In another preferred embodiment the invention relates to crystalline tiotropium malate, in particular to crystalline tiotropium L-malate, preferably a crystalline solvated form containing N,N-dimethylamide (=DMA) of tiotropium L-malate, which is characterized by an monoclinic elementary cell. In yet another preferred embodiment the invention relates to crystalline tiotropium L-malate DMA solvate which is characterized by a monoclic elementary cell with the parameters a=7.4670(5)Å, b=14.4950(9)Å, c=14.0490(14)Å, β=100.095(2)° and cell volume=1497.0(2) Å³ determined by single crystal X-ray structural analysis. This malate form is optionally also referred to as malate form I.

In yet another preferred embodiment the invention relates to a crystalline solvate containing 1-methyl-2-pyrrolidinone (=NMP) of tiotropium L-malate which is characterized by a X-ray powder diagram with the characteristic values d=9.97 Å; 6.47 Å; 5.92 Å; 5.25 Å; 5.12 Å; 5.09 Å; 3.51 Å; inter alia. This malate form is optionally also referred to as malate form II.

In another preferred embodiment the invention relates to crystalline tiotropium succinate, in particular to crystalline tiotropium succinate which is characterized by a X-ray powder diagram with the characteristic values d=7.90 Å; 4.73 Å; 4.08 Å; 3.93 Å; 3.17 Å; inter alia. In another aspect the present invention relates to a method of preparing the new crystalline tiotropium succinate which is explained by way of example in the experimental section that follows.

In another preferred embodiment the invention relates to crystalline tiotropium malonate, in particular to crystalline tiotropium malonate which is characterized by a X-ray powder diagram with the characteristic values d=6.67 Å; 5.14 Å; 4.92 Å; 4.57 Å; 4.39 Å; 3.32 Å; inter alia. In another aspect the present invention relates to a method of preparing the new crystalline tiotropium malonate which is explained by way of example in the experimental section that follows.

In another preferred embodiment the invention relates to crystalline tiotropium tartrate, in particular to crystalline tiotropium tartrate which is characterized by a X-ray powder diagram with the characteristic values d=20.16 Å; 5.46 Å; 5.08 Å; 4.41 Å; inter alia. This tartrate form is optionally also referred to as tartrate form I.

In yet another preferred embodiment the invention relates to crystalline tiotropium tartrate which is characterized by a X-ray powder diagram with the characteristic values d=15.71 Å; 9.38 Å; 5.26 Å; 4.88 Å; 4.11 Å; inter alia. This tartrate form is optionally also referred to as tartrate form II.

In yet another preferred embodiment the invention relates to crystalline tiotropium tartrate which is characterized by a X-ray powder diagram with the characteristic values d=5.19 Å; 5.04 Å; 4.05 Å; inter alia. This tartrate form is optionally also referred to as tartrate form III.

In another aspect the present invention relates to methods of preparing the new crystalline tiotropium tartrate forms which are explained by way of example in the experimental section that follows.

In another preferred embodiment the invention relates to crystalline tiotropium oxalate, in particular to crystalline tiotropium oxalate dihydrate, preferably crystalline tiotropium oxalate dihydrate which is characterized by a monoclinic elementary cell. In yet another preferred embodiment the invention relates to crystalline tiotropium oxalate dihydrate which is characterized by a monoclinic elementary cell with the parameters a=11.4540(4)Å, b=10.0620(4)Å, c=20.2480(9)Å, β=95.969(2)°, and cell volume=2320.93(16) Å³ determined by single crystal X-ray structural analysis. This oxalate form is optionally also referred to as oxalate form I. In another aspect the present invention relates to a method of preparing the new crystalline tiotropium oxalate dihydrate which is explained by way of example in the experimental section that follows.

In another preferred embodiment the invention relates to crystalline, anhydrous tiotropium p-toluenesulphonate (tosylate), which is characterized by a X-ray powder diagram with the characteristic values d=5.08 Å; 4.48 Å; 3.84 Å; inter alia. This p-toluenesulphonate form is optionally also referred to as p-toluenesulphonate form II. Tiotropium p-toluenesulphonate form I is known from WO05/042528. In another aspect the present invention relates to a method of preparing the new crystalline tiotropium p-toluenesulphonate which is explained by way of example in the experimental section that follows.

In another preferred embodiment the invention relates to crystalline tiotropium methanesulphonate monohydrate, in particular to crystalline tiotropium methanesulphonate monohydrate which is characterized by a X-ray powder diagram with the characteristic values d=4.47 Å; 4.09 Å; 3.79 Å; 3.54 Å; inter alia. This methanesulphonate form is optionally also referred to as methanesulphonate form II. The anhydrous tiotropium methanesulphonate form I is known from WO05/042528. In another aspect the present invention relates to a method of preparing the new crystalline tiotropium p-toluenesulphonate which is explained by way of example in the experimental section that follows.

The novel tiotropium forms can be used for preparing a pharmaceutical composition for the treatment of obstructive pulmonary diseases selected from among bronchial asthma, paediatric asthma, severe asthma, acute asthma attacks, chronic bronchitis, and COPD while it is particularly preferable according to the invention to use them for preparing a pharmaceutical composition for the treatment of bronchial asthma.

It is also preferable to use the novel tiotropium forms for preparing a pharmaceutical composition for the treatment of pulmonary emphysema which has its origins in COPD (chronic obstructive pulmonary disease) or al-proteinase inhibitor deficiency.

It is also preferable to use the novel tiotropium forms for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use novel tiotropium forms for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the novel tiotropium forms for preparing a pharmaceutical composition for the treatment of cystic fibrosis or mucoviscidosis.

It is also preferable to use novel tiotropium forms for preparing a pharmaceutical composition for the treatment of bronchitis, such as for example bronchitis caused by bacterial or viral infection, allergic bronchitis and toxic bronchitis.

It is also preferable to use the novel tiotropium forms for preparing a pharmaceutical composition for the treatment of bronchiectasis.

It is also preferable to use the novel tiotropium forms for preparing a pharmaceutical composition for the treatment of ARDS (adult respiratory distress syndrome).

It is also preferable to use the novel tiotropium forms for preparing a pharmaceutical composition for the treatment of pulmonary oedema, for example toxic pulmonary oedema after aspiration or inhalation of toxic substances and foreign substances.

It is particularly preferable to use the novel tiotropium forms detailed above for preparing a pharmaceutical composition for the treatment of asthma or COPD. Also of particular importance is the above-mentioned use of the novel tiotropium forms for preparing a pharmaceutical composition for once-a-day treatment of inflammatory and obstructive respiratory complaints, particularly for the once-a-day treatment of asthma.

The present invention also relates to a process for the treatment of the above-mentioned diseases, characterised in that one or more of the above-mentioned novel tiotropium forms are administered in therapeutically effective amounts. The present invention further relates to processes for the treatment of the aforementioned diseases, characterised in that one or more of the above-mentioned novel tiotropium forms are administered once a day in therapeutically effective amounts. In the treatment of the aforementioned respiratory diseases the new tiotropium modifications disclosed herein are preferably administered via formulations that are suitable for inhalation. In formulations dedicated for administration via inhalation the physicochemical properties of a drug substance may influence decisively stability, usefulness and efficacy of the formulation. In this respect the novel tiotropium salts show advantageous properties not yet disclosed in the art.

The present invention also relates to new pharmaceutical formulations which contain the above-mentioned new tiotropium salts. This may be done using inhalable powdered formulations, propellant-containing aerosol formulations or propellant-free inhalable solutions.

The present invention also relates to inhalable powder containing 0.001 to 3% tiotropium in the form of tiotropium salts mentioned hereinbefore with a physiologically acceptable excipient. By tiotropium is meant the ammonium cation.

Inhalable powders which contain 0.01 to 2% tiotropium are preferred according to the invention. Particularly preferred inhalable powders contain tiotropium in an amount from about 0.03 to 1%, preferably 0.05 to 0.6%, particularly preferably 0.06 to 0.3%. Of particular importance according to the invention, finally, are inhalable powders which contain about 0.08 to 0.22% tiotropium. The amounts of tiotropium specified above are based on the amount of tiotropium cation contained.

The excipients that are used for the purposes of the present invention are prepared by suitable grinding and/or screening using current methods known in the art. The excipients used according to the invention may also be mixtures of excipients which are obtained by mixing excipient fractions of different mean particle sizes.

Examples of physiologically acceptable excipients which may be used to prepare the inhalable powders used to produce the inhalable powders for use in the inhalettes according to the invention include monosaccharides (e.g. glucose, fructose or arabinose), disaccharides (e.g. lactose, saccharose, maltose, trehalose), oligo- and polysaccharides (e.g. dextrans, dextrins, maltodextrin, starch, cellulose), polyalcohols (e.g. sorbitol, mannitol, xylitol), cyclodextrins (e.g. α-cyclodextrin, β-cyclodextrin, χ-cyclodextrin, methyl-β-cyclodextrin, hydroxypropyl-β-cyclodextrin), amino acids (e.g. arginine hydrochloride) or salts (e.g. sodium chloride, calcium carbonate), or mixtures thereof. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. The average particle size may be determined using methods known in the art (cf. for example WO 02/30389, paragraphs A and C). Finally, in order to prepare the inhalable powder according to the invention, micronised tiotropium salt, which is preferably characterised by an average particle size of 0.5 to 10 μm, particularly preferably from 1 to 5 μm, is added to the excipient mixture. The average particle size may be determined using methods known in the art (cf. for example WO 02/30389, paragraph B). Processes for grinding and micronising active substances are known from the prior art.

If no specifically prepared excipient mixture is used as the excipient, it is particularly preferable to use excipients which have a mean particle size of 10-50 μm and a 10% fine content.

By average particle size is meant here the 50% value of the volume distribution measured with a laser diffractometer using the dry dispersion method. The average particle size may be determined using methods known in the art (cf. for example WO 02/30389, paragraphs A and C). Analogously, the 10% fine content in this instance refers to the 10% value of the volume distribution measured using a laser diffractometer. In other words, for the purposes of the present invention, the 10% fine content denotes the particle size below which 10% of the quantity of particles is found (based on the volume distribution).

The percentages given within the scope of the present invention are always percent by weight, unless specifically stated to the contrary.

In particularly preferred inhalable powders the excipient is characterised by a mean particle size of 12 to 35 μm, particularly preferably from 13 to 30 μm.

Also particularly preferred are those inhalable powders wherein the 10% fine content is about 1 to 4 μm, preferably about 1.5 to 3 μm.

The inhalable powders according to the invention are characterised, in accordance with the problem on which the invention is based, by a high degree of homogeneity in the sense of the accuracy of single doses. This is in the region of <8%, preferably <6%, most preferably <4%.

After the starting materials have been weighed out the inhalable powders are prepared from the excipient and the active substance using methods known in the art. Reference may be made to the disclosure of WO 02/30390, for example. The inhalable powders according to the invention may accordingly be obtained by the method described below, for example. In the preparation methods described hereinafter the components are used in the proportions by weight described in the above-mentioned compositions of the inhalable powders.

First, the excipient and the active substance are placed in a suitable mixing container. The active substance used has an average particle size of 0.5 to 10 μm, preferably 1 to 6 μm, most preferably 2 to 5 μm. The excipient and the active substance are preferably added using a sieve or a granulating sieve with a mesh size of 0.1 to 2 mm, preferably 0.3 to 1 mm, most preferably 0.3 to 0.6 mm Preferably, the excipient is put in first and then the active substance is added to the mixing container. During this mixing process the two components are preferably added in batches. It is particularly preferred to sieve in the two components in alternate layers. The mixing of the excipient with the active substance may take place while the two components are still being added. Preferably, however, mixing is only done once the two components have been sieved in layer by layer.

The present invention also relates to the use of the inhalable powders according to the invention for preparing a pharmaceutical composition for the treatment of respiratory diseases as outlined in more detail hereinbefore.

The inhalable powders according to the invention may for example be administered using inhalers which meter a single dose from a reservoir by means of a measuring chamber (e.g. according to U.S. Pat. No. 4,570,630A) or by other means (e.g. according to DE 36 25 685 A). Preferably, however, the inhalable powders according to the invention are packed into capsules (to make so-called inhalettes), which are used in inhalers such as those described in WO 94/28958, for example.

Capsules containing the inhalable powder according to the invention may for instance be administered using an inhaler as shown in FIG. 1 of WO 03/084502. This inhaler is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut and airholes 13 for adjusting the flow resistance.

The present invention further relates to the use of the inhalable powders according to the invention for preparing a pharmaceutical composition for treating respiratory complaints, characterised in that the inhaler described above is used.

For administering the inhalable powders according to the invention using powder-filled capsules it is particularly preferred to use capsules the material of which is selected from among the synthetic plastics, most preferably selected from among polyethylene, polycarbonate, polyester, polypropylene and polyethylene terephthalate. Particularly preferred synthetic plastic materials are polyethylene, polycarbonate or polyethylene terephthalate. If polyethylene is used as one of the capsule materials which is particularly preferred according to the invention, it is preferable to use polyethylene with a density of between 900 and 1000 kg/m³, preferably 940-980 kg/m³, more preferably about 960-970 kg/m³ (high density polyethylene).

The synthetic plastics according to the invention may be processed in various ways using manufacturing methods known in the art. Injection moulding of the plastics is preferred according to the invention. Injection moulding without the use of mould release agents is particularly preferred. This method of production is well defined and is characterised by being particularly reproducible.

In another aspect the present invention relates to the abovementioned capsules which contain the abovementioned inhalable powders according to the invention. These capsules may contain about 1 to 20 mg, preferably about 3 to 15 mg, most preferably about 4 to 12 mg of inhalable powder. Preferred formulations according to the invention contain 4 to 6 mg of inhalable powder. Of equivalent importance according to the invention are capsules for inhalation which contain the formulations according to the invention in an amount of from 8 to 12 mg.

The present invention also relates to an inhalation kit consisting of one or more of the above capsules characterised by a content of inhalable powder according to the invention in conjunction with the inhaler according to FIG. 1 in WO 03/084502.

The present invention also relates to the use of the abovementioned capsules characterised by a content of inhalable powder according to the invention, for preparing a pharmaceutical composition for treating respiratory complaints.

Filled capsules which contain the inhalable powders according to the invention are produced by methods known in the art, by filling the empty capsules with the inhalable powders according to the invention.

In another preferred embodiment according to the invention the inhaler according to U.S. Pat. No. 4,524,769 is applied. This inhaler (or inhalator) is activated by the air flow generated at inhalation. The disclosure of U.S. Pat. No. 4,524,769 is incorporated herein by reference in its entirety. Accordingly, in a preferred embodiment the invention relates to a method for the administration of an inhalable powder containing a novel tiotropium salt 1, preferably in an amount of 0.001 to 5 tiotropium, in admixture with a physiologically acceptable excipient with an average particle size of between 10 to 500 μm, by means of the inhaler according to U.S. Pat. No. 4,524,769, comprising a nozzle, a conduit connected to the nozzle, a storage chamber adjacent said conduit for storing said inhalable powder to be dispensed by said inhalator, a perforated membrane having a plurality of preselected perforated portions each holding and dispensing a reproducible unit dose of less than 50 mg of the said inhalable powder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber, dose loading means for introducing said inhalable powder in the storage chamber into the perforation of the preselected portion of said membrane disposed within the storage chamber, and maneuvering means for displacing the perforated membrane through a plurality of positions whereby successive preselected portions of the perforated membrane holding the inhalable powder are positioned across said conduit for dispensing the inhalable powder.

In another embodiment, the invention relates to a method for treatment of respiratory diseases, characterized in that an inhalable powder containing a novel tiotropium salt 1, preferably in an amount of 0.001 to 5 tiotropium, in admixture with a physiologically acceptable excipient with an average particle size of between 10 to 500 μm, is administered via inhalation by the inhaler according to U.S. Pat. No. 4,524,769, comprising a nozzle, a conduit connected to the nozzle, a storage chamber adjacent said conduit for storing said inhalable powder to be dispensed by said inhalator, a perforated membrane having a plurality of preselected perforated portions each holding and dispensing a reproducible unit dose of less than 50 mg of the said inhalable powder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber, dose loading means for introducing said inhalable powder in the storage chamber into the perforation of the preselected portion of said membrane disposed within the storage chamber, and maneuvering means for displacing the perforated membrane through a plurality of positions whereby successive preselected portions of the perforated membrane holding the inhalable powder are positioned across said conduit for dispensing the inhalable powder.

In another preferred embodiment the invention relates to the use of the inhaler according to U.S. Pat. No. 4,524,769 comprising a nozzle, a conduit connected to the nozzle, a storage chamber adjacent said conduit for storing said inhalable powder to be dispensed by said inhalator, a perforated membrane having a plurality of preselected perforated portions each holding and dispensing a reproducible unit dose of less than 50 mg of the said inhalable powder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber, dose loading means for introducing said inhalable powder in the storage chamber into the perforation of the preselected portion of said membrane disposed within the storage chamber, and maneuvering means for displacing the perforated membrane through a plurality of positions whereby successive preselected portions of the perforated membrane holding the inhalable powder are positioned across said conduit for dispensing the inhalable powder, for the administration of an inhalable powdered containing a novel tiotropium salt 1, preferably in an amount of 0.001 to 5% tiotropium, in admixture with a physiologically acceptable excipient with an average particle size of between 10 to 500 μm.

In yet another preferred embodiment the invention relates to an inhalation kit consisting of an inhalable powdered containing a novel tiotropium salt 1, preferably in an amount of 0.001 to 5 tiotropium, in admixture with a physiologically acceptable excipient with an average particle size of between 10 to 500 μm, and the inhaler according to U.S. Pat. No. 4,524,769, comprising a nozzle, a conduit connected to the nozzle, a storage chamber adjacent said conduit for storing said inhalable powder to be dispensed by said inhalator, a perforated membrane having a plurality of preselected perforated portions each holding and dispensing a reproducible unit dose of less than 50 mg of the said inhalable powder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber, dose loading means for introducing said inhalable powder in the storage chamber into the perforation of the preselected portion of said membrane disposed within the storage chamber, and maneuvering means for displacing the perforated membrane through a plurality of positions whereby successive preselected portions of the perforated membrane holding the inhalable powder are positioned across said conduit for dispensing the inhalable powder.

In another preferred embodiment according to the invention the inhaler according to U.S. Pat. No. 5,590,645 is applied. The disclosure of U.S. Pat. No. 5,590,645 is incorporated herein by reference in its entirety. Accordingly, in a preferred embodiment the invention relates to a method for the administration of an inhalable powder containing a novel tiotropium salt 1, preferably in an amount of 0.001 to 5 tiotropium, in admixture with a physiologically acceptable excipient with an average particle size of between 10 to 500 μm, by means of the inhaler according to 5,590,645, comprising a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and indexing means for indexing in communication with said outlet containers of a medicament pack in use with said inhalation device.

In another embodiment, the invention relates to a method for treatment of respiratory diseases, characterized in that an inhalable powder containing a novel tiotropium salt 1, preferably in an amount of 0.001 to 5 tiotropium, in admixture with a physiologically acceptable excipient with an average particle size of between 10 to 500 μm, is administered via inhalation by the inhaler according to U.S. Pat. No. 5,590,645, comprising a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and indexing means for indexing in communication with said outlet containers of a medicament pack in use with said inhalation device.

In another preferred embodiment the invention relates to the use of the inhaler according to U.S. Pat. No. 5,590,645, comprising a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and indexing means for indexing in communication with said outlet containers of a medicament pack in use with said inhalation device, for the administration of an inhalable powder containing a novel tiotropium salt 1, preferably in an amount of 0.001 to 5% tiotropium, in admixture with a physiologically acceptable excipient with an average particle size of between 10 to 500 μm.

In yet another preferred embodiment the invention relates to an inhalation kit consisting of an inhalable powder containing a novel tiotropium salt 1, preferably in an amount of 0.001 to 5 tiotropium, in admixture with a physiologically acceptable excipient with an average particle size of between 10 to 500 μm, and the inhaler according to U.S. Pat. No. 5,590,645, comprising a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and indexing means for indexing in communication with said outlet containers of a medicament pack in use with said inhalation device.

In another preferred embodiment according to the invention the inhaler according to U.S. Pat. No. 4,627,432 is applied. The disclosure of U.S. Pat. No. 4,627,432 is incorporated herein by reference in its entirety. Accordingly, in a preferred embodiment the invention relates to a method for the administration of an inhalable powder c containing a novel tiotropium salt 1, preferably in an amount of 0.001 to 5 tiotropium, in admixture with a physiologically acceptable excipient with an average particle size of between 10 to 500 μm, by means of the inhaler according to U.S. Pat. No. 4,627,432, being characterised by a housing with a chamber therein, an air inlet into the chamber, a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening.

In another embodiment, the invention relates to a method for treatment of respiratory diseases, characterized in that an inhalable powder containing a novel tiotropium salt 1, preferably in an amount of 0.001 to 5 tiotropium, in admixture with a physiologically acceptable excipient with an average particle size of between 10 to 500 μm, is administered via inhalation by the inhaler according to U.S. Pat. No. 4,627,432, being characterised by a housing with a chamber therein, an air inlet into the chamber, a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening.

In another preferred embodiment the invention relates to the use of the inhaler according to U.S. Pat. No. 4,627,432 being characterised by a housing with a chamber therein, an air inlet into the chamber, a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening, for the administration of an inhalable powder containing a novel tiotropium salt 1, preferably in an amount of 0.001 to 5 tiotropium, in admixture with a physiologically acceptable excipient with an average particle size of between 10 to 500 μm.

In yet another preferred embodiment the invention relates to an inhalation kit consisting of an inhalable powder containing a novel tiotropium salt 1, preferably in an amount of 0.001 to 5 tiotropium, in admixture with a physiologically acceptable excipient with an average particle size of between 10 to 500 μm, and the inhaler according to U.S. Pat. No. 4,627,432, being characterised by a housing with a chamber therein, an air inlet into the chamber, a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening.

Moreover, the inhalable powders comprising one of the novel tiotropium salts 1 according to the invention may be administered also by use of an inhaler selected from among the following devices, known in the art: Spinhaler, Rotahaler, Easyhaler, Novolizer, Clickhaler, Pulvinal, Twisthaler or for example Jethaler.

The new tiotropium salts may optionally also be administered in the form of propellant-containing inhalable aerosols. Aerosol formulations in the form of solutions or suspensions may be used for this.

The term aerosol solution denotes pharmaceutical formulations in which the new tiotropium salt and any excipients used are completely dissolved.

The present invention provides aerosol formulations containing the new tiotropium salt 1, which contain in addition to one of the above-mentioned tiotropium salts an HFA propellant, a co-solvent and an inorganic or organic acid and which are further characterised in that the concentration of the acid is such that in aqueous solution it corresponds to a pH in the range from 2.5-4.5.

The above-mentioned aerosol solutions are characterised by a particularly high stability.

Preferred aerosol solutions are characterised in that the concentration of the acid is such that in aqueous solution it corresponds to a pH in the range from 3.0-4.3, particularly preferably from 3.5-4.0.

The aerosol solutions according to the invention may also contain a small amount of water (preferably up to 5%, particularly preferably up to 3%, more preferably up to 2%).

The aerosol solutions according to the invention preferably contain an amount of novel tiotropium salt 1 such that the proportion of tiotropium cation they contain is between 0.00008 and 0.4%, preferably between 0.0004 and 0.16%, particularly preferably between 0.0008 and 0.08%.

Suitable HFA propellants within the scope of the aerosol solutions are those which form a homogeneous propellant formulation with the co-solvents used, in which a therapeutically effective amount of the tiotropium salt 1 may be dissolved. Preferred HFA propellants according to the invention are propellants selected from the group consisting of 1,1,1,2-tetrafluoroethane (HFA-134(a)), 1,1,1,2,3,3,3,-heptafluoropropane (HFA-227), HFA-32 (difluoromethane), HFA-143(a) (1.1.1-trifluoroethane), HFA-134 (1,1,2,2-tetrafluoroethane) and HFA-152a (1,1-difluoroethane. HFA-134(a) and HFA-227 are particularly preferred according to the invention, while HFA-134(a) is particularly important according to the invention. In addition to the HFA propellants mentioned above, non-halogenated propellants may also be used on their own or mixed with one or more of the above-mentioned HFA propellants. Examples of such non-halogenated propellants are saturated hydrocarbons such as for example n-propane, n-butane or isobutane, or also ethers such as diethyl ether, for example.

Organic or inorganic acids may be used as acids according to the invention. Inorganic acids within the scope of the present invention are selected for example from the group consisting of hydrochloric acid, sulphuric acid, nitric acid or phosphoric acid, while according to the invention it is preferable to use hydrochloric or sulphuric acid, particularly hydrochloric acid. Organic acids within the scope of the present invention are selected for example from the group consisting of ascorbic acid, citric acid, lactic acid, maleic acid, benzoic acid or tartaric acid, while ascorbic acid and citric acid are preferred according to the invention.

The aerosol solutions according to the invention may be obtained analogously to methods known in the art.

Pharmaceutically acceptable excipients may optionally be contained in the aerosol solutions according to the invention. For example, soluble surfactants and lubricants may be used. Examples of such soluble surfactants and lubricants include sorbitan trioleate, lecithin or isopropyl myristate. Other excipients which may be present may be antioxidants (for example ascorbic acid or tocopherol), flavour masking agents (for example menthol, sweeteners and synthetic or natural flavourings).

Examples of co-solvents which may be used according to the invention are alcohols (for example ethanol, isopropanol and benzylalcohol), glycols (for example propyleneglycol, polyethyleneglycols, polypropyleneglycol, glycolether, block copolymers of oxyethylene and oxypropylene) or other substances such as for example glycerol, polyoxyethylene alcohols, polyoxyethylene fatty acid esters and glycofurols (such as for example glycofurol 75). A preferred co-solvent according to the invention is ethanol.

The amount of co-solvents which may be used in the formulations according to the invention is preferably in the range from 5-50%, preferably 10-40%, particularly preferably 15-30% based on the total formulation.

Unless stated to the contrary, the percentages specified within the scope of the present invention are to be read as percent by weight.

The formulations according to the invention may contain small amounts of water, as already mentioned previously. In a preferred aspect, the present invention relates to formulations in which the content of water is up to 5%, particularly preferably up to 3%, more preferably up to 2%.

In another aspect the present invention relates to aerosol solutions which contain no water. In these formulations the amount of cosolvent is preferably in the range from 20-50%, preferably in the range from 30-40%.

The formulations according to the invention may be administered using inhalers known in the art (pMDIs=pressurized metered dose inhalers).

The present invention also relates to the use of the above-mentioned aerosol solutions characterised by a content of a novel tiotropium salt 1 according to the invention for preparing a pharmaceutical composition for the treatment of respiratory complaints and diseases.

The present invention also relates to suspensions of the novel tiotropium salts 1 according to the invention in the propellant gases HFA 227 and/or HFA 134a, optionally combined with one or more other propellant gases, preferably selected from the group consisting of propane, butane, pentane, dimethylether, CHClF₂, CH₂F₂, CF₃CH₃, isobutane, isopentane and neopentane.

According to the invention those suspensions which contain as propellant gas only HFA 227, a mixture of HFA 227 and HFA 134a or only HFA 134a are preferred.

If a mixture of the propellent gases HFA 227 and HFA 134a is used in the suspension formulations according to the invention, the weight ratios in which these two propellent gas components are used are freely variable.

If one or more other propellent gases, selected from the group consisting of propane, butane, pentane, dimethylether, CHClF₂, CH₂F₂, CF₃CH₃, isobutane, isopentane and neopentane are used in addition to the propellent gases HFA 227 and/or HFA 134a in the suspension formulations according to the invention, the amount of this additional propellent gas component is preferably less than 50%, preferably less than 40%, particularly preferably less than 30%.

The suspensions according to the invention preferably contain an amount of the novel tiotropium forms such that the amount of tiotropium cation is between 0.001 and 0.8%, preferably between 0.08 and 0.5%, and particularly preferably between 0.2 and 0.4% according to the invention.

Unless stated to the contrary, the percentages given within the scope of the present invention are always percent by weight.

In some cases, the term suspension formulation is used within the scope of the present invention instead of the term suspension. The two terms are to be regarded as equivalent within the scope of the present invention.

The propellant-containing inhalable aerosols or suspension formulations according to the invention may also contain other constituents such as surface-active agents (surfactants), adjuvants, antioxidants or flavourings.

The surface-active agents (surfactants) optionally present in the suspensions according to the invention are preferably selected from the group consisting of Polysorbate 20, Polysorbate 80, Myvacet 9-45, Myvacet 9-08, isopropyl myristate, oleic acid, propyleneglycol, polyethyleneglycol, Brij, ethyl oleate, glyceryl trioleate, glyceryl monolaurate, glyceryl monooleate, glyceryl monostearate, glyceryl monoricinoleate, cetylalcohol, sterylalcohol, cetylpyridinium chloride, block polymers, natural oil, ethanol and isopropanol. Of the above-mentioned suspension adjuvants Polysorbate 20, Polysorbate 80, Myvacet 9-45, Myvacet 9-08 or isopropyl myristate are preferably used. Myvacet 9-45 or isopropyl myristate are most preferably used.

If the suspensions according to the invention contain surfactants these are preferably used in an amount of 0.0005-1%, particularly preferably 0.005-0.5%.

The adjuvants optionally contained in the suspensions according to the invention are preferably selected from the group consisting of alanine, albumin, ascorbic acid, aspartame, betaine, cysteine, phosphoric acid, nitric acid, hydrochloric acid, sulphuric acid and citric acid. Ascorbic acid, phosphoric acid, hydrochloric acid or citric acid are preferably used, while hydrochloric acid or citric acid is most preferably used.

If adjuvants are present in the suspensions according to the invention, these are preferably used in an amount of 0.0001-1.0%, preferably 0.0005-0.1%, particularly preferably 0.001-0.01%, while an amount of 0.001-0.005% is particularly important according to the invention.

The antioxidants optionally contained in the suspensions according to the invention are preferably selected from the group consisting of ascorbic acid, citric acid, sodium edetate, editic acid, tocopherols, butylhydroxytoluene, butylhydroxyanisol and ascorbylpalmitate, while tocopherols, butylhydroxytoluene, butylhydroxyanisol or ascorbylpalmitate are preferably used.

The flavourings optionally contained in the suspensions according to the invention are preferably selected from the group consisting of peppermint, saccharine, Dentomint, aspartame and ethereal oils (for example cinnamon, aniseed, menthol, camphor), peppermint or Dentomint® being particularly preferred.

With a view to administration by inhalation it is essential to provide the active substances in finely divided form. For this purpose, the novel tiotropium forms according to the invention are either ground (micronised) or obtained in finely divided form by other technical processes known in principle from the prior art (for example precipitation, spray drying). Methods of micronising active substances are known in the art. Preferably after micronising the active substance has a mean particle size of 0.5 to 10 μm, preferably 1 to 6 μm, particularly preferably 1.5 to 5 μm auf. Preferably at least 50%, preferably at least 60%, particularly preferably at least 70% of the particles of active substance have a particle size which is within the size ranges mentioned above. Particularly preferably at least 80%, most preferably at least 90% of the particles of active substance have a particle size which is within the size ranges mentioned above.

The suspensions according to the invention may be prepared using methods known in the art. For this, the constituents of the formulation are mixed with the propellent gas or gases (optionally at low temperatures) and filled into suitable containers.

The above-mentioned propellant-containing suspensions according to the invention may be administered using inhalers known in the art (pMDIs=pressurized metered dose inhalers). Accordingly, in another aspect, the present invention relates to pharmaceutical compositions in the form of suspensions as hereinbefore described combined with one or more inhalers suitable for administering these suspensions. Moreover the present invention relates to inhalers, characterised in that they contain the propellant-containing suspensions according to the invention described hereinbefore.

The present invention also relates to containers (cartridges) which when fitted with a suitable valve can be used in a suitable inhaler and which contain one of the above-mentioned propellant-containing suspensions according to the invention. Suitable containers (cartridges) and processes for filling these cartridges with the propellant-containing suspensions according to the invention are known in the art.

In view of the pharmaceutical activity of tiotropium the present invention also relates to the use of the suspensions according to the invention for preparing a pharmaceutical composition for inhalation or nasal administration, preferably for preparing a pharmaceutical composition for inhalative or nasal treatment of diseases in which anticholinergics may develop a therapeutic benefit.

Particularly preferably the present invention also relates to the use of the suspensions according to the invention for preparing a pharmaceutical composition for the inhalative treatment of respiratory complaints, preferably asthma or COPD.

The new tiotropium forms may optionally also be administered in the form of propellant-free inhalable aerosols. For administering these propellant-free inhalable aerosols the new tiotropium forms are prepared in the form of pharmaceutical solutions.

The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The preferred solvent is water without the addition of ethanol.

The concentration of the novel tiotropium forms according to the invention based on the amount of tiotropium in the finished pharmaceutical preparation depends on the therapeutic effect desired. For the majority of complaints that respond to tiotropium the concentration of tiotropium is between 0.0005 and 5 wt. %, preferably between 0.001 and 3 wt. %.

The pH of the formulation according to the invention is between 2.0 and 4.5, preferably between 2.5 and 3.5 and more preferably between 2.7 and 3.3 and particularly preferably between 2.7 and 3.2. Most preferred are pH values with an upper limit of 3.1.

The pH is adjusted by the addition of pharmacologically acceptable acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with the active substance. Of the organic acids, ascorbic acid, fumaric acid and citric acid are preferred. If desired, mixtures of the above acids may be used, particularly in the case of acids which have other properties in addition to their acidifying qualities, e.g. as flavourings or antioxidants, such as citric acid or ascorbic acid, for example. Hydrochloric acid is expressly mentioned as an inorganic acid.

Pharmacologically acceptable bases may also be used, if desired, for precisely titrating the pH. Suitable bases include for example alkali metal hydroxides and alkali metal carbonates. The preferred alkali metal ion is sodium. When such bases are used, care must be taken to ensure that the salts resulting from them which are then contained in the finished pharmaceutical formulation are also pharmacologically compatible with the above-mentioned acid.

According to the invention, the addition of editic acid (EDTA) or one of the known salts thereof, sodium edetate, as stabiliser or complexing agent is unnecessary in the present formulation.

Another embodiment contains editic acid and/or the above-mentioned salts thereof.

In a preferred embodiment the content based on sodium edetate is less than 10 mg/100 ml. In this case one preferred range is between 5 mg/100 ml and less than 10 mg/100 ml and another is between more than 0 and 5 mg/100 ml.

In another embodiment the content of sodium edetate is from 10 up to 30 mg/100 ml, and is preferably not more than 25 mg/100 ml.

In a preferred embodiment this additive is omitted altogether.

The remarks made above for sodium edetate also apply analogously to other comparable additives which have complexing properties and may be used instead of it, such as for example nitrilotriacetic acid and the salts thereof.

By complexing agents are preferably meant within the scope of the present invention molecules which are capable of entering into complex bonds. Preferably, these compounds should have the effect of complexing cations, most preferably metal cations.

In addition to ethanol, other co-solvents and/or other excipients may also be added to the formulation according to the invention.

Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters, provided that they are not also the solvent or suspension agent.

The terms excipients and additives in this context denote any pharmacologically acceptable and therapeutically beneficial substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as sorbitan trioleate, polyvinylpyrrolidone, other stabilisers, complexing agents, antioxidants and/or preservatives which prolong the shelf life of the finished pharmaceutical formulation, flavourings, vitamins and/or other additives known in the art. The additives also include pharmacologically acceptable salts such as sodium chloride.

The preferred excipients include antioxidants such as ascorbic acid, for example, provided that it has not already been used to adjust the pH, vitamin A, vitamin E, tocopherols and similar vitamins or provitamins occurring in the human body.

Preservatives may be used to protect the formulation from contamination with pathogens. Suitable preservatives are those which are known in the art, particularly benzalkonium chloride or benzoic acid or benzoates such as sodium benzoate in the concentration known from the prior art.

Preferred formulations contain, in addition to the solvent water and one of the novel tiotropium forms only benzalkonium chloride and sodium edetate. In another preferred embodiment, no sodium edetate is present.

The solutions according to the invention are preferably administered using the Respimat® inhaler. A more advance embodiment of this inhaler is disclosed in WO 97/12687 and FIGS. 6 therein.

If the new tiotropium forms according to the invention are used for the manufacture of a medicament for the treatment of one or several respiratory diseases outlined hereinbefore, it may also be advantageous to combine them with one or two additional active compounds. These additional active compounds may be selected from among betamimetics 2a, EGFR inhibitors 2b, PDEIV-inhibitors 2c, steroids 2d, and LTD4 antagonists 2e, optionally together with a pharmaceutically acceptable excipient.

Within the instant application the term betamimetic is optionally also replaced by the term beta₂-agonist. According to the instant invention preferred beta₂ agonists 2a are selected from the group consisting of albuterol (2a.1), bambuterol (2a.2), bitolterol (2a.3), broxaterol (2a.4), carbuterol (2a.5), clenbuterol (2a.6), fenoterol (2a.7), formoterol (2a.8), hexoprenaline (2a.9), ibuterol (2a.10), isoetharine (2a.11), isoprenaline (2a.12), levosalbutamol (2a.13), mabuterol (2a.14), meluadrine (2a.15), metaproterenol (2a.16), orciprenaline (2a.17), pirbuterol (2a.18), procaterol (2a.19), reproterol (2a.20), TD 3327 (2a.21), ritodrine (2a.22), salmeterol (2a.23), salmefamol (2a.24), soterenot (2a.25), sulphonterol (2a.26), tiaramide (2a.27), terbutaline (2a.28), tolubuterol (2a.29), CHF-4226 (=TA 2005 or carmoterol; 2a.30), HOKU-81 (2a.31), KUL-1248 (2a.32), 3-(4-{6-[2-Hydroxy-2-(4-hydroxy-3-hydroxymethyl-phenyl)-ethylamino]-hexyloxy}-butyl)-benzenesulfoneamide (2a.33), 5-[2-(5,6-Diethyl-indan-2-ylamino)-1-hydroxy-ethyl]-8-hydroxy-1H-quinolin-2-one (2a.34), 4-hydroxy-7-[2-{[2-{[3-(2-phenylethoxy)propyl]sulphonyl}ethyl]-amino}ethyl]-2(3H)-benzothiazolone (2a.35), 1-(2-fluoro-4-hydroxyphenyl)-2-[4-(1-benzimidazolyl)-2-methyl-2-butylamino]ethanol (2a.36), 1-[3-(4-methoxybenzyl-amino)-4-hydroxyphenyl]-2-[4-(1-benzimidazolyl)-2-methyl-2-butylamino]ethanol (2a.37), 1-[2H-5-hydroxy-3-oxo-4H-1,4-benzoxazin-8-yl]-2-[3-(4-N,N-dimethylaminophenyl)-2-methyl-2-propylamino]ethanol (2a.38), 1-[2H-5-hydroxy-3-oxo-4H-1,4-benzoxazin-8-yl]-2-[3-(4-methoxyphenyl)-2-methyl-2-propylamino]ethanol (2a.39), 1-[2H-5-hydroxy-3-oxo-4H-1,4-benzoxazin-8-yl]-2-[3-(4-n-butyloxyphenyl)-2-methyl-2-propylamino]ethanol (2a.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (2a.41), 5-hydroxy-8-(1-hydroxy-2-isopropylaminobutyl)-2H-1,4-benzoxazin-3-(4H)-one (2a.42), 1-(4-amino-3-chloro-5-trifluormethylphenyl)-2-tert.-butylamino)ethanol (2a.43), 1-(4-ethoxycarbonylamino-3-cyano-5-fluorophenyl)-2-(tert.-butylamino)ethanol (2a.44), and N-[2-Hydroxy-5-(1-hydroxy-2-{2-[4-(2-hydroxy-2-phenyl-ethylamino)-phenyl]-ethylamino}-ethyl)-phenyl]-formamide (2a.45), optionally in the form of the racemates, the enantiomers, the diastereomers and optionally the pharmacologically acceptable acid addition salts and the hydrates thereof.

According to the instant invention more preferred beta₂ agonists 2a are selected from the group consisting of bambuterol (2a.2), bitolterol (2a.3), carbuterol (2a.5), clenbuterol (2a.6), fenoterol (2a.7), formoterol (2a.8), hexoprenaline (2a.9), ibuterol (2a.10), pirbuterol (2a.18), procaterol (2a.19), reproterol (2a.20), TD 3327 (2a.21), salmeterol (2a.23), sulphonterol (2a.26), terbutaline (2a.28), tolubuterol (2a.29), CHF-4226 (=TA 2005 or carmoterol; 2a.30), 3-(4-{6-[2-Hydroxy-2-(4-hydroxy-3-hydroxymethyl-phenyl)-ethylamino]-hexyloxy}-butyl)-benzenesulfoneamide (2a.33), 5-[2-(5,6-Diethyl-indan-2-ylamino)-1-hydroxy-ethyl]-8-hydroxy-1H-quinolin-2-one (2a.34), 4-hydroxy-7-[2-{[2-{[3-(2-phenylethoxy)propyl]sulphonyl}ethyl]-amino}ethyl]-2(3H)-benzothiazolone (2a.35), 1-(2-fluoro-4-hydroxyphenyl)-2-[4-(1-benzimidazolyl)-2-methyl-2-butylamino]ethanol (2a.36), 1-[3-(4-methoxybenzyl-amino)-4-hydroxyphenyl]-2-[4-(1-benzimidazolyl)-2-methyl-2-butylamino]ethanol (2a.37), 1-[2H-5-hydroxy-3-oxo-4H-1,4-benzoxazin-8-yl]-2-[3-(4-N,N-dimethylaminophenyl)-2-methyl-2-propylamino]ethanol (2a.38), 1-[2H-5-hydroxy-3-oxo-4H-1,4-benzoxazin-8-yl]-2-[3-(4-methoxyphenyl)-2-methyl-2-propylamino]ethanol (2a.39), 1-[2H-5-hydroxy-3-oxo-4H-1,4-benzoxazin-8-yl]-2-[3-(4-n-butyloxyphenyl)-2-methyl-2-propylamino]ethanol (2a.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (2a.41), 5-hydroxy-8-(1-hydroxy-2-isopropylaminobutyl)-2H-1,4-benzoxazin-3-(4H)-one (2a.42), 1-(4-amino-3-chloro-5-trifluormethylphenyl)-2-tert.-butylamino)ethanol (2a.43), 1-(4-ethoxycarbonylamino-3-cyano-5-fluorophenyl)-2-(tert.-butylamino)ethanol (2a.44), and N-[2-Hydroxy-5-(1-hydroxy-2-{2-[4-(2-hydroxy-2-phenyl-ethylamino)-phenyl]-ethylamino}-ethyl)-phenyl]-formamide (2a.45), optionally in the form of the racemates, the enantiomers, the diastereomers and optionally the pharmacologically acceptable acid addition salts and the hydrates thereof.

More preferably, the betamimetics 2a are selected from the group consisting of fenoterol (2a.7), formoterol (2a.8), salmeterol (2a.23), CHF-4226 (=TA 2005 or carmoterol; 2a.30), 3-(4-{6-[2-Hydroxy-2-(4-hydroxy-3-hydroxymethyl-phenyl)-ethylamino]-hexyloxy}-butyl)-benzenesulfoneamide (2a.33), 5-[2-(5,6-Diethyl-indan-2-ylamino)-1-hydroxy-ethyl]-8-hydroxy-1H-quinolin-2-one (2a.34), 1-[3-(4-methoxybenzyl-amino)-4-hydroxyphenyl]-2-[4-(1-benzimidazolyl)-2-methyl-2-butylamino]ethanol (2a.37), 1-[2H-5-hydroxy-3-oxo-4H-1,4-benzoxazin-8-yl]-2-[3-(4-N,N-dimethylaminophenyl)-2-methyl-2-propylamino]ethanol (2a.38), 1-[2H-5-hydroxy-3-oxo-4H-1,4-benzoxazin-8-yl]-2-[3-(4-methoxyphenyl)-2-methyl-2-propylamino]ethanol (2a.39), 1-[2H-5-hydroxy-3-oxo-4H-1,4-benzoxazin-8-yl]-2-[3-(4-n-butyloxyphenyl)-2-methyl-2-propylamino]ethanol (2a.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (2a.41), and N-[2-Hydroxy-5-(1-hydroxy-2-{2-[4-(2-hydroxy-2-phenyl-ethylamino)-phenyl]-ethylamino}-ethyl)-phenyl]-formamide (2a.45), optionally in the form of the racemates, the enantiomers, the diastereomers and optionally the pharmacologically acceptable acid addition salts and the hydrates thereof. Of the betamimetics mentioned above the compounds formoterol (2a.8), salmeterol (2a.23), CHF-4226 (=TA 2005 or carmoterol; 2a.30), 3-(4-{6-[2-Hydroxy-2-(4-hydroxy-3-hydroxymethyl-phenyl)-ethylamino]-hexyloxy}-butyl)-benzenesulfoneamide (2a.33), 5-[2-(5,6-Diethyl-indan-2-ylamino)-1-hydroxy-ethyl]-8-hydroxy-1H-quinolin-2-one are (2a.34), and N-[2-Hydroxy-5-(1-hydroxy-2-{2-[4-(2-hydroxy-2-phenyl-ethylamino)-phenyl]-ethylamino}-ethyl)-phenyl]-formamide (2a.45), particularly preferred, optionally in the form of the racemates, the enantiomers, the diastereomers and optionally the pharmacologically acceptable acid addition salts thereof, and the hydrates thereof.

Examples of pharmacologically acceptable acid addition salts of the betamimetics 2a according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, 4-phenylcinnamic acid, 5-(2.4-difluorophenyl)salicylic acid or maleic acid. If desired, mixtures of the abovementioned acids may also be used to prepare the salts 2a.

According to the invention, the salts of the betamimetics 2a selected from among the hydrochloride, hydrobromide, sulphate, phosphate, fumarate, methanesulphonate, 4-phenylcinnamate, 5-(2.4-difluorophenyl)salicylate, maleate and xinafoate are preferred. Particularly preferred are the salts of 2a in the case of salmeterol selected from among the hydrochloride, sulphate, 4-phenylcinnamate, 5-(2.4-difluorophenyl)salicylate and xinafoate, of which the 4-phenylcinnamate, 5-(2.4-difluorophenyl)salicylate and especially xinafoate are particularly important. Particularly preferred are the salts of 2a in the case of formoterol selected from the hydrochloride, sulphate, hemifumarate and fumarate, of which the hydrochloride, hemifumarate and fumarate are particularly preferred. Of exceptional importance according to the invention is formoterol fumarate dihydrate or formoterol hemifumarate hydrate.

Any reference to the term betamimetics 2a also includes a reference to the relevant enantiomers or mixtures thereof.

In the pharmaceutical compositions according to the invention, the compounds 2a may to be present in the form of their racemates, enantiomers or mixtures thereof. The separation of the enantiomers from the racemates may be carried out using methods known in the art (e.g. by chromatography on chiral phases, etc.) If the compounds 2a are used in the form of their enantiomers, it is particularly preferable to use the enantiomers in the R configuration at the C—OH group. If the compounds 2a possess 2 chiral carbon atoms they are preferably used in the form of their pure diastereomers, particularly in the form of those diasteromers that possess R configuration at the C—OH group. An example may be R,R-formoterol.

The EGFR inhibitor 2b is preferably selected from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxyl-7-methoxy-quinazoline, 4-[(3-chloro-4-fluorophenyl)amino]-6-({[4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl]amino}-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-[(R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-64(8)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetyl amino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2.2.1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, cetuximab, trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

The EGFR-inhibitor 2b is preferably selected from among the 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[44(R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxyl-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-∂(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-[(R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-{(3-chloro-4-fluoro-phenyl)amino}-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetyl amino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl}-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2.2.1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and cetuximab, optionally in the form of the racemates, enantiomers or diastereomers thereof, optionally in the form of the pharmacologically acceptable acid addition salts thereof, the solvates and/or hydrates thereof.

Particularly preferably, the EGFR-inhibitors 2b are selected from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxyl-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxyl-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-[(R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxyl-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonyl amino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methane sulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

Particularly preferred medicament combinations according to the invention contain as EGFR-inhibitors 2b those compounds which are selected from the group comprising
4-[(3-chloro-4-fluorophenyl)amino]-6-{[4-(morpholin-4-yl)-1-oxo-2-buten-1-yl]-amino}-7-cyclopropylmethoxy-quinazoline (2b.1),
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline (2b.2),
4-[(3-chloro-4-fluoro-phenyl)amino]-6-[2-((S)-6-methyl-2-oxo-morpholin-4-yl)-ethoxy]-7-methoxy-quinazoline (2b.3),
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline (2b A),
4-[(3-ethynyl-phenyl)amino]-6,7-bis-(2-methoxy-ethoxy)-quinazoline (2b.5),
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline (2b.6),
4-[(3-ethynyl-phenyl)amino]-6-{[4-(5,5-dimethyl-2-oxo-morpholin-4-yl)-1-oxo-2-buten-1-yl]amino}-quinazoline (2b.7),
4-[(3-chloro-4-fluoro-phenyl)amino]-6-(trans-4-methanesulphonylamino-cyclohexan-1-yloxy)-7-methoxy-quinazoline (2b.8),
4-[(3-chloro-4-fluoro-phenyl)amino]-6-(tetrahydropyran-3-yloxy)-7-methoxy-quinazoline (2b.9),
4-[(3-chloro-4-fluoro-phenyl)amino]-6-{1-[(morpholin-4-yl)carbonyl]-piperidin-4-yloxy}-7-methoxy-quinazoline (2b.10),
4-[(3-chloro-4-fluoro-phenyl)amino]-6-{1-[2-(2-oxopyrrolidin-1-yl)ethyl]-piperidin-4-yloxy}-7-methoxy-quinazoline (2b.11),
4-[(3-ethynyl-phenyl)amino]-6-(1-acetyl-piperidin-4-yloxy)-7-methoxy-quinazoline (2b.12),
4-[(3-ethynyl-phenyl)amino]-6-(1-methyl-piperidin-4-yloxy)-7-methoxy-quinazoline (2b.13),
4-[(3-ethynyl-phenyl)amino]-6-(1-methanesulphonyl-piperidin-4-yloxy)-7-methoxy-quinazoline (2b.14),
4-[(3-ethynyl-phenyl)amino]-6-{1-[(morpholin-4-yl)carbonyl]-piperidin-4-yloxy}-7-methoxy-quinazoline (2b.15),
4-[(3-chloro-4-fluoro-phenyl)amino]-6-{1-[(2-methoxyethyl)carbonyl]-piperidin-4-yloxy}-7-methoxy-quinazoline (2b.16),
4-[(3-chloro-4-fluoro-phenyl)amino]-6-[cis-4-(N-methanesulphonyl-N-methyl-amino)-cyclohexan-1-yloxy]-7-methoxy-quinazoline (2b.17),
4-[(3-chloro-4-fluoro-phenyl)amino]-6-[cis-4-(N-acetyl-N-methyl-amino)-cyclohexan-1-yloxy]-7-methoxy-quinazoline (2b.18),
4-[(3-chloro-4-fluoro-phenyl)amino]-6-(trans-4-methylamino-cyclohexan-1-yloxy)-7-methoxy-quinazoline (2b.19),
4-[(3-chloro-4-fluoro-phenyl)amino]-6-[trans-4-(N-methanesulphonyl-N-methyl-amino)-cyclohexan-1-yloxy]-7-methoxy-quinazoline (2b.20),
4-[(3-chloro-4-fluoro-phenyl)amino]-6-(trans-4-dimethylamino-cyclohexan-1-yloxy)-7-methoxy-quinazoline (2b.21),
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline (2b.22),
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline (2b.23),
4-[(3-chloro-4-fluoro-phenyl)amino]-6-(1-methanesulphonyl-piperidin-4-yloxy)-7-methoxy-quinazoline (2b.24) and
4-[(3-chloro-4-fluoro-phenyl)amino]-6-(1-cyano-piperidin-4-yloxy)-7-methoxy-quinazoline (2b.25),

optionally in the form of the racemates, enantiomers or diastereomers thereof, optionally in the form of the pharmacologically acceptable acid addition salts thereof, the solvates and/or hydrates thereof.

By the acid addition salts with pharmacologically acceptable acids which the compounds 2b may possibly be capable of forming are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

The PDE IV-inhibitor 2c is preferably selected from among enprofyllin (2c.1), theophyllin (2c.2), roflumilast (2c.3), ariflo (Cilomilast, 2c.4)), CP-325,366 (2c.5), BY343 (2c.6), D-4396 (Sch-351591, 2c.7)), AWD-12-281 (GW-842470, 3)), N-(3,5-dichloro-1-oxo-pyridin-4-yl)-4-difluoromethoxy-3-cyclopropylmethoxybenzamide (2c.9), NCS-613 (2c.10), pumafentine (2c.11), (+)p-R4aR*,10bS*)-9-ethoxy-1,2,3,4,4a,10b-hexahydro-8-methoxy-2-methylbenzo[s][1,6]naphthyridin-6-yl]-N,N-diisopropylbenzamide (2c.12), (R)-(+)-1-(4-bromobenzyl)-4-[(3-cyclopentyloxy)-4-methoxyphenyl]-2-pyrrolidone (2c.13), 3-(cyclopentyloxy-4-methoxyphenyl)-1-(4-N′-[N-2-cyano-S-methyl-isothioureido]benzyl)-2-pyrrolidone (2c.14), cis[4-cyano-4-(3-cyclopentyloxy-4-methoxyphenyl)cyclohexane-1-carboxylic acid] (2c.15), 2-carbomethoxy-4-cyano-4-(3-cyclopropylmethoxy-4-difluoromethoxyphenyl)cyclohexan-1-one (2c.16), cis[4-cyano-4-(3-cyclopropylmethoxy-4-difluoromethoxyphenyl)cyclohexan-1-ol] (2c.17), (R)-(+)-ethyl[4-(3-cyclopentyloxy-4-methoxyphenyl)pyrrolidin-2-ylidene]acetate (2c.18), (S)-(−)-ethyl[4-(3-cyclopentyloxy-4-methoxyphenyl)pyrrolidin-2-ylidene]acetate (2c.19), 4-(3-cyclopentyloxy-4-methoxy-phenyl)-3-(1-hydroxy-ethyl)-3-methyl-pyrrolidine-1-carboxylic acid methyl ester (=IC 485, 2c.20), CDP840 (2c.21), Bay-198004 (2c.22), D-4418 (2c.23), PD-168787 (2c.24), T-440 (2c.25), T-2585 (2c.26), arofyllin (2c.27), atizoram (2c.28), V-11294A (2c.29), Cl-1018 (2c.30), CDC-801 (2c.31), CDC-3052 (2c.32), D-22888 (2c.33), YM-58997 (2c.34), Z-15370 (2c.35), 9-cyclopentyl-5,6-dihydro-7-ethyl-3-(2-thienyl)-9H-pyrazolo[3,4-c]-1,2,4-triazolo[4,3-a]pyridine (2c.36), 9-cyclopentyl-5,6-dihydro-7-ethyl-3-(tert-butyl)-9H-pyrazolo[3,4-c]-1,2,4-triazolo[4,3-a]pyridine (2c.37), and tetomilast (2c.38), optionally in the form of the racemates, enantiomers or diastereomers thereof and optionally in the form of the pharmacologically acceptable acid addition salts, solvates and/or hydrates thereof.

In particularly preferred medicament combinations the PDE IV-inhibitor 2c is selected from the group comprising enprofyllin (2c.1), roflumilast (2c.3) optionally also in form of the roflumilast N-oxide, ariflo (cilomilast) (2c.4), AWD-12-281 (GW-842470) (2c.8), N-(3,5-dichloro-1-oxo-pyridin-4-yl)-4-difluoromethoxy-3-cyclopropylmethoxybenzamide (2c.9), T-440 (2c.25), T-2585 (2c.26), cis[4-cyano-4-(3-cyclopentyloxy-4-methoxyphenyl)cyclohexane-1-carboxylic acid] (2c.15), 2-carbomethoxy-4-cyano-4-(3-cyclopropylmethoxy-4-difluoromethoxyphenyl)cyclohexan-1-one (2c.16), cis[4-cyano-4-(3-cyclopropylmethoxy-4-difluoromethoxyphenyl)cyclohexan-1-ol] (2c.17), 4-(3-cyclopentyloxy-4-methoxy-phenyl)-3-(1-hydroxy-ethyl)-3-methyl-pyrrolidine-1-carboxylic acid methyl ester (=IC 485, 2c.20), PD-168787 (2c.24), arofyllin (2c.27), atizoram (2c.28), V-11294A (2c.29), C1-1018 (2c.30), CDC-801 (2c.31), D-22888 (2c.33), YM-58997 (2c.34), Z-15370 (2c.35), 9-cyclopentyl-5,6-dihydro-7-ethyl-3-(2-thienyl)-9H-pyrazolo[3,4-c]-1,2,4-triazolo[4,3-a]pyridine (2c.36), 9-cyclopentyl-5,6-dihydro-7-ethyl-3-(tert-butyl)-9H-pyrazolo[3,4-c]-1,2,4-triazolo[4,3-a]pyridine (2c.37), and tetomilast (2c.38), optionally in the form of the racemates, enantiomers or diastereomers thereof and optionally in the form of the pharmacologically acceptable acid addition salts, solvates and/or hydrates thereof.

In particularly preferred medicament combinations the PDE IV-inhibitor 2c is selected from the group comprising roflumilast (2c.3), ariflo (cilomilast) (2c.4), AWD-12-281 (GW-842470) (2c.8), 2-carbomethoxy-4-cyano-4-(3-cyclopropylmethoxy-4-difluoromethoxyphenyl)cyclohexan-1-one (2c.16), cis[4-cyano-4-(3-cyclopropylmethoxy-4-difluoromethoxyphenyl)cyclohexan-1-ol] (2c.17), 4-(3-cyclopentyloxy-4-methoxy-phenyl)-3-(1-hydroxy-ethyl)-3-methyl-pyrrolidine-1-carboxylic acid methyl ester (=IC 485, 2c.20), arofyllin (2c.27), atizoram (2c.28), Z-15370 (2c.35), 9-cyclopentyl-5,6-dihydro-7-ethyl-3-(2-thienyl)-9H-pyrazolo[3,4-c]-1,2,4-triazolo[4,3-a]pyridine (2c.36), 9-cyclopentyl-5,6-dihydro-7-ethyl-3-(tert-butyl)-9H-pyrazolo[3,4-c]-1,2,4-triazolo[4,3-a]pyridine (2c.37), and tetomilast (2c.38), while roflumilast (2c.3), Z-15370 (2c.35) and AWD-12-281 (2c.8) are of particular significance, optionally in the form of the racemates, enantiomers or diastereomers thereof and optionally in the form of the pharmacologically acceptable acid addition salts, solvates and/or hydrates thereof.

By the acid addition salts with pharmacologically acceptable acids which the compounds 2c may possibly be capable of forming are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Other preferred medicament combinations according to the invention contain as an additional active substance, in addition to one or more, preferably one compound 1, one or more, preferably one steroid 2d, optionally in combination with pharmaceutically acceptable excipients.

In such medicament combinations the steroid 2d is preferably selected from among prednisolone (2d.1), prednisone (2d.2), butixocortpropionate (2d.3), RPR-106541 (2d.4), flunisolide (2d.5), beclomethasone (2d.6), triamcinolone (2d.7), budesonide (2d.8), fluticasone (2d.9), mometasone (2d.10), ciclesonide (2d.11), rofleponide (2d.12), ST-126 (2d.13), dexamethasone (2d.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2d.15), (S)-(2-oxo-tetrahydro-furan-3S-yl)6α,9α-difluoro-11β-hydroxy-16α-methyl-3-oxo-17α-propionyloxy-androsta-1,4-diene-17β-carbothionate (2d.16) and etiprednol-dichloroacetate (BNP-166, 2d.17), optionally in the form of the racemates, enantiomers or diastereomers thereof and optionally in the form of the salts and derivatives thereof, the solvates and/or hydrates thereof.

In particularly preferred medicament combinations the steroid 2d is selected from the group comprising flunisolide (2d.5), beclomethasone (2d.6), triamcinolone (2d.7), budesonide (2d.8), fluticasone (2d.9), mometasone (2d.10), ciclesonide (2d.11), rofleponide (2d.12), ST-126 (2d.13), dexamethasone (2d.14), (S)-fluoromethyl 6α,9α-difluoro-17α-[2-furanylcarbonyl)oxy]-11β-hydroxy-16α-methyl-3-oxo-androsta-1,4-diene-17β-carbothionate (2d.15), (S)-(2-oxo-tetrahydro-furan-3S-yl)6α,9α-difluoro-11β-hydroxy-16α-methyl-3-oxo-17α-propionyloxy-androsta-1,4-diene-17β-carbothionate (2d.16) and etiprednol-dichloroacetate (2d.17), optionally in the form of the racemates, enantiomers or diastereomers thereof and optionally in the form of the salts and derivatives thereof, the solvates and/or hydrates thereof.

In particularly preferred medicament combinations the steroid 2d is selected from the group comprising budesonide (2d.8), fluticasone (2c), mometasone (2d.10), ciclesonide (2d.11), (S)-fluoromethyl 6α,9α-difluoro-17α-[2-furanylcarbonyl)oxy]-11β-hydroxy-16α-methyl-3-oxo-androsta-1,4-diene-17β-carbothionate (2d.15) and etiprednol-dichloroacetate (2d.17), optionally in the form of the racemates, enantiomers or diastereomers thereof and optionally in the form of the salts and derivatives thereof, the solvates and/or hydrates thereof.

Any reference to steroids 2d includes a reference to any salts or derivatives, hydrates or solvates thereof which may exist. Examples of possible salts and derivatives of the steroids 2d may be: alkali metal salts, such as for example sodium or potassium salts, sulphobenzoates, phosphates, isonicotinates, acetates, propionates, dihydrogen phosphates, palmitates, pivalates or furoates.

Other preferred medicament combinations according to the invention contain, as an additional active substance, in addition to one or more, preferably one compound 1, one or more, preferably one, LTD4 antagonist 2e, optionally in combination with pharmaceutically acceptable excipients.

In such medicament combinations the LTD4 antagonist 2e is preferably selected from among montelukast (2e.1), 1-(((R)-(3-(2-(6,7-difluoro-2-quinolinyl)ethenyl)phenyl)-3-(2-(2-hydroxy-2-propyl)phenyl)thio)methylcyclopropane-acetic acid (2e.2), 1-(((1(R)-3(3-(2-(2,3-dichlorothieno[3,2-b]pyridin-5-yI)-(E)-ethenyl)phenyl)-3-(2-(1-hydroxy-1-methylethyl)phenyl)propyl)thio)methyl)cyclopropanacetic acid (2e.3), pranlukast (2e.4), zafirlukast (2e.5), [2-[[2-(4-tert-butyl-2-thiazolyl)-5-benzofuranyl]oxymethyl]-phenyl]acetic acid (2e.6), MCC-847 (ZD-3523) (2e.7), MN-001 (3), MEN-91507 (LM-1507) (2e.9), VUF-5078 (2e.10), VUF-K-8707 (2e.11) and L-733321 (2e.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In preferred medicament combinations the LTD4 antagonist 2e is selected from the group comprising montelukast (2e.1), pranlukast (2e.4), zafirlukast (2e.5) MCC-847 (ZD-3523) (2e.7), MN-001 (2e.8), MEN-91507 (LM-1507) (2e.9), VUF-5078 (2e.10), VUF-K-8707 (2e.11) and L-733321 (2e.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In particularly preferred medicament combinations the LTD4 antagonist 2e is selected from the group comprising montelukast (2e.1), pranlukast (2e.4), zafirlukast (2e.5), MCC-847 (ZD-3523) (2e.7), MN-001 (2e.8) and MEN-91507 (LM-1507) (2e.9), while montelukast (2e.1), pranlukast (2e.4) and zafirlukast (2e.5) are particularly preferred, optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

By the acid addition salts with pharmacologically acceptable acids which the compounds 2e may possibly be capable of forming are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of possible salts and derivatives which the compounds 2e may possibly be capable of forming include for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

Surprisingly, the novel tiotropium forms do furthermore display extremely promising properties in the treatment of other diseases that have not yet been disclosed in the art for tiotropium forms already known. These surprising findings are outlined in more detail below.

The use of acetylcholine esterase inhibitors as contact insecticides is—despite of the remarkable toxicological properties of these chemical structures for humans and animals—worldwide distributed Important members of this compound class are for example esters of phosphoric acid, esters of tiophosphoric acid and esters of carbamic acid. In addition to their use as insecticides e.g. in agriculture, acetylcholine esterase inhibitors have also been developed as chemical weapons. Examples are: sarin, soman, tabun. These compounds exert a pronounced toxicity (LD50 for soman: 0.14 mg/kg per os). The mode of action is a difficult to reverse inhibition of acetylcholine esterase via phosphorylation of the aminoacid serin located in the erster centrum of the enzyme. An intoxication with acetylcholine esterase inhibitors induces the following typical symptoms: Nausea, vomiting, diarrhea, hidrosis, miosis, salivation, increased secretion in bronchi, bronchoconstriction, bradycardia and, sometimes, ventricular fibrillation. Furthermore, beside these peripheral symptoms, the following central nervous symptoms occur: anxiety, headache, seizures and paralysis of the respiratory muscles. Surprisingly it can be found that tiotropium is useful for the treatment of the peripheral symptoms of intoxication with acetylcholine esterase inhibitors—if not contra-indicated. Surprisingly, a long duration of action of tiotropium in this new indication is observed. Therefore, the present invention refers to the use of tiotropium salts of formula 1 for the manufacture of a medicament for the treatment of peripheral symptoms of an intoxication with acetylcholine esterase inhibitors. In particular, the present invention refers to the use of the novel tiotropium forms according to the invention for the manufacture of a medicament for the treatment of peripheral symptoms of an intoxication with acetylcholine esterase inhibitors. In another embodiment the invention relates to a process for the treatment of peripheral symptoms of an intoxication with acetylcholine esterase inhibitors characterised in that one or more of the above-mentioned novel tiotropium forms are administered in therapeutically effective amounts. The present invention further relates to processes for the treatment of peripheral symptoms of an intoxication with acetylcholine esterase inhibitors, characterised in that one or more of the above-mentioned novel tiotropium forms are administered once a day in therapeutically effective amounts.

Furthermore, it can surprisingly be found that tiotropium is useful to induce a vagal blockade during long lasting interventions performed under anesthesia—if not contra-indicated. Surprisingly, a long duration of action of tiotropium in this new indication is observed. Therefore, the present invention refers to the use of tiotropium salts for the manufacture of a medicament for anesthesia premedication inducing a long lasting blockade of the vagal reflexes. In particular, the present invention refers to the use of the novel tiotropium forms according to the invention for the manufacture of a medicament for anesthesia premedication inducing a long lasting blockade of the vagal reflexes. In another embodiment the invention relates to a process to induce a long lasting blockade of the vagal reflexes, characterised in that one or more of the above-mentioned novel tiotropium forms are administered in therapeutically effective amounts. The present invention further relates to processes to induce a long lasting blockade of the vagal reflexes, characterised in that one or more of the above-mentioned novel tiotropium forms are administered once a day in therapeutically effective amounts.

Furthermore, it can surprisingly be found that tiotropium is useful for the blockade of the vagal stimulation in conditions associated with gastrointestinal spasms and gastrointestinal hypermotility symptoms—if not contra-indicated. Surprisingly, a long duration of action of tiotropium in this new indication is observed. Therefore, the present invention refers to the use of tiotropium salts for the manufacture of a medicament for the treatment of spasms as well as hypermotility of the gastro-intestinal tract resulting in a normalization of the motility disorder. In particular, the present invention refers to the use of the novel tiotropium forms according to the invention for the manufacture of a medicament for the treatment of spasms as well as hypermotility of the gastro-intestinal tract resulting in a normalization of the motility disorder. In another embodiment the invention relates to a process for blocking of the vagal stimulation in conditions associated with gastrointestinal spasms and gastrointestinal hypermotility symptoms, characterised in that one or more of the above-mentioned novel tiotropium forms are administered in therapeutically effective amounts. The present invention further relates to processes for blocking of the vagal stimulation in conditions associated with gastrointestinal spasms and gastrointestinal hypermotility symptoms, characterised in that one or more of the above-mentioned novel tiotropium forms are administered once a day in therapeutically effective amounts.

Furthermore, it can surprisingly be found that tiotropium has an anti-proliferative mode of action. Accordantly, the present invention refers to the use of tiotropium for the manufacture of a medicament for the treatment or prevention, preferred for the treatment of proliferative processes and diseases. Preferably, the present invention relates to the use of the above-mentioned novel tiotropium forms for preparing a pharmaceutical composition for the prevention or treatment, preferably the treatment, of proliferative processes in cell types selected from among the fibroblasts, myofibroblasts, epithelial cells, endothelial cells, serous and mucosal cells in submucosal glands, Clara cells, type I+II pneumocytes and goblet cells. Preferably the present invention relates to the above-mentioned use of the above-mentioned novel tiotropium forms for preparing a pharmaceutical composition for the prevention or treatment, preferably the treatment, of proliferative processes which occur in diseases of the upper and lower respiratory organs including the lungs. Preferably the present invention relates to the above-mentioned use of the above-mentioned novel tiotropium forms for preparing a pharmaceutical composition for the prevention or treatment, preferably the treatment, of proliferative processes which occur in diseases selected from the group consisting of lung inflammation, pulmonary hypertension, pulmonary emphysema, pulmonary fibrosis, pulmonary oedema, bronchiectasis, Adult Respiratory Distress Syndrome (ARDS), Boeck's disease, fibrosing alveolitis, pulmonary embolism, pneumoconiosis (e.g. asbestosis, silicosis), lung cancer and tuberculosis. Preferably the present invention relates to the above-mentioned use of the above-mentioned novel tiotropium forms for preparing a pharmaceutical composition for the treatment of proliferative processes which occur in diseases selected from the group consisting of pulmonary inflammation, pulmonary hypertension, pulmonary emphysema, pulmonary oedema, Adult Respiratory Distress Syndrome (ARDS), Boeck's disease, pulmonary fibrosis, pulmonary embolism, pneumoconiosis (e.g. asbestosis, silicosis), lung cancer and tuberculosis. Preferably the present invention relates to the above-mentioned use of the above-mentioned novel tiotropium forms for preparing a pharmaceutical composition for the prevention or treatment, preferably the treatment, of proliferative processes which occur in pulmonary inflammation. Moreover the present invention relates to the use of the above-mentioned novel tiotropium forms for preparing a pharmaceutical composition for the prevention or treatment, preferably for the treatment of pulmonary inflammation. Preferably the present invention relates to the above-mentioned use of the above-mentioned novel tiotropium forms for preparing a pharmaceutical composition for the prevention or treatment, preferably the treatment, of proliferative processes which occur in pulmonary hypertension. Moreover the present invention relates to the use of the above-mentioned novel tiotropium forms for preparing a pharmaceutical composition for the prevention or treatment, preferably for the treatment of pulmonary hypertension. Preferably the present invention relates to the above-mentioned use of the above-mentioned novel tiotropium forms for preparing a pharmaceutical composition for the prevention or treatment, preferably the treatment, of proliferative processes which occur in pulmonary emphysema. Moreover the present invention relates to the use of the above-mentioned novel tiotropium forms for preparing a pharmaceutical composition for the prevention or treatment, preferably for the treatment of pulmonary emphysema. Preferably the present invention relates to the above-mentioned use of the above-mentioned novel tiotropium forms for preparing a pharmaceutical composition for the prevention or treatment, preferably the treatment, of proliferative processes which occur in pulmonary oedemas. Moreover the present invention relates to the use of the above-mentioned novel tiotropium forms for preparing a pharmaceutical composition for the prevention or treatment, preferably for the treatment of pulmonary oedema.

Preferably the present invention relates to the above-mentioned use of the above-mentioned novel tiotropium forms for preparing a pharmaceutical composition for the prevention or treatment, preferably the treatment, of proliferative processes which occur in ARDS. Moreover the present invention relates to the use of the above-mentioned novel tiotropium forms for preparing a pharmaceutical composition for the prevention or treatment, preferably for the treatment of ARDS. Preferably the present invention relates to the above-mentioned use of the above-mentioned novel tiotropium forms for preparing a pharmaceutical composition for the prevention or treatment, preferably the treatment, of proliferative processes which occur in Boeck's disease. Moreover the present invention relates to the use of the above-mentioned novel tiotropium forms for preparing a pharmaceutical composition for the prevention or treatment, preferably for the treatment of Boeck's disease. Preferably the present invention relates to the above-mentioned use of the above-mentioned novel tiotropium forms for preparing a pharmaceutical composition for the prevention or treatment, preferably the treatment, of proliferative processes which occur in pulmonary fibrosis. Moreover the present invention relates to the use of the above-mentioned novel tiotropium forms for preparing a pharmaceutical composition for the prevention or treatment, preferably for the treatment of pulmonary fibrosis. Preferably the present invention relates to the above-mentioned use of the above-mentioned novel tiotropium forms for preparing a pharmaceutical composition for the prevention or treatment, preferably the treatment, of proliferative processes which occur in pulmonary embolism. Moreover the present invention relates to the use of the above-mentioned novel tiotropium forms for preparing a pharmaceutical composition for the prevention or treatment, preferably for the treatment of pulmonary embolism. Preferably the present invention relates to the above-mentioned use of the above-mentioned novel tiotropium forms for preparing a pharmaceutical composition for the prevention or treatment, preferably the treatment, of proliferative processes which occur in pneumoconiosis (e.g. asbestosis, silicosis). Moreover the present invention relates to the use of the above-mentioned novel tiotropium forms for preparing a pharmaceutical composition for the prevention or treatment, preferably for the treatment of pneumoconiosis (e.g. asbestosis, silicosis). Preferably the present invention relates to the above-mentioned use of the above-mentioned novel tiotropium forms for preparing a pharmaceutical composition for the prevention or treatment, preferably the treatment, of proliferative processes which occur in lung cancer. Moreover the present invention relates to the use of the above-mentioned novel tiotropium forms for preparing a pharmaceutical composition for the prevention or treatment, preferably for the treatment of lung cancer.

Preferably the present invention relates to the above-mentioned use of the above-mentioned novel tiotropium forms for preparing a pharmaceutical composition for the prevention or treatment, preferably the treatment, of proliferative processes which occur in tuberculosis. Moreover the present invention relates to the use of the above-mentioned novel tiotropium forms for preparing a pharmaceutical composition for the prevention or treatment, preferably for the treatment of tuberculosis. In another aspect the present invention relates to pharmaceutical formulations containing an novel tiotropium forms for the treatment of the above-mentioned diseases. Moreover the present invention relates to the use of formulations containing an novel tiotropium forms for preparing a pharmaceutical composition for the prevention or treatment, preferably for the treatment of the above-mentioned diseases.

For the therapy of the aforementioned diseases the novel tiotropium forms may also be administered via inhalation. However, other administration modes can also be taken into account. Suitable preparations for administering the novel tiotropium forms include tablets, capsules, suppositories, suspensions, solutions, patches etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers. Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets. Syrups or elixirs containing the active substances or combinations of active substances according to the invention may additionally contain a sweetener such as saccharine, cyclamate, glycerol or sugar and a flavour enhancer, e.g. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates. Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles. Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

The Examples that follow serve to illustrate the present invention still further, without restricting the scope of the invention to the embodiments by way of example that follow.

### EXAMPLES OF SYNTHESIS ACCORDING TO THE INVENTION AND CHARACTERIZATION OF PRODUCTS

Methods:

Single Crystal X-Ray Diffraction

Suitable single crystal selected after the crystallization experiments, were glued to a glass fibre, which was mounted on an X-ray diffraction goniometer. X-ray diffraction data was collected for these crystals at a temperature of 233 K using a KappaCCD system and MoKα radiation generated by a FR590 X-ray generator (Bruker Nonius, Delft, The Netherlands).

Unit-cell parameters and crystal structures were determined and refined using the software package maXus (Mackay et al., 1997). From the crystal structure the theoretical X-ray powder diffraction pattern were calculated using PowderCell for Windows version 2.3 (Kraus et al., 1999).

X-Ray Powder Diffraction

X-Ray powder diffraction patterns were obtained using Avantium's T2 high throughput XRPD set-up. The plates were mounted on a Bruker GADDS diffractometer that is equipped with a Hi-Star area detector. The XRPD platform is calibrated using Silver Behenate for the long d-spacings and Corundum for the short d-spacings.

The data collection was carried out at room temperature using monochromatic CuK_{α} radiation in the region of 2θ between 1.5 and 41.5°. The diffraction pattern of each well was collected in two 2theta ranges (1.5≧2θ≦19.5° for the 1st frame, and 21.5≦2θ≦41.5° for the second frame) with an exposure time between 90 and 180 s for each frame. No background subtraction or curve smoothing was applied to the XRPD patterns.

Melting Points—Differential Scanning Calorimetry

Melting properties were obtained from differential scanning calorimetry (DSC) thermograms, recorded with a DSC822e (Mettler-Toledo GmbH, Switzerland). The DSC822e is calibrated for temperature and enthalpy with a small piece of indium (m.p.=156.6° C.; ΔHf=28.45 J.g⁻¹). Samples are sealed in standard 40 μl aluminum pans and heated in the DSC from 25 to 300° C. with a heating rate of 10 and 20° C. min⁻¹ Dry N₂ gas is used to purge the DSC equipment during measurement at a flow rate of 50 ml min⁻¹

Conditioning of Ion Exchange Resin:

For the preparation of the different salt forms of tiotropium an anionic ion exchange resin (=IER) was used. Prior to all preparations the following way of conditioning was applied to the IER:

A column (length: 1.5 m; diameter: 4 cm) was charged with 3×100 g of Ion Exchange Resin (IER) (Dowex 1×8-200 Cl, Aldrich 21,742-5, Lot: 01428JB, 1.5 eq/ml). The column was rinsed with 41 of a saturated aqueous NaHCO₃ solution (Aldrich 34,094-4, Lot: 505422-203 in demineralised water) with a flow of ca. 15 ml/min. The completion of the exchange was checked by taking a 2 ml sample of the elute that was acidified with a 1 M HNO3 (aq) solution and adding 2 drops of a 0.5 M AgNO₃ (aq) solution to the stirred solution. A precipitate indicates the presence of CP ions while a clear solution indicates completion of the exchange. Next, the column was rinsed with demineralised water until the pH was essentially neutral (pH=6-7) and identical to demineralised water. Approximately 91 of water was required to achieve this (flow 15 ml/min) The IER was transferred to an Erlenmeyer flask and was kept as slurry.

Preparation of Tiotropium Bicarbonate 2:

A glass column (ca. 110 ml) was filled with the dry ion exchange resin and afterwards filled with deionized water. The column was loaded with bicarbonate using a saturated NaHCO3-solution (ca. 90 g NaHCO3/1). About 4.5 l of this saturated NaHCO3-solution were passed through the column with a flow rate of approx. 10 ml/min Afterwards the column was washed with 61 of deionized water (flow rate: 20 ml/min) to get rid of excess of NaHCO3. 50 ml of a tiotropiumbromide solution (conc. 10 mg/ml) are slowly (flow rate: 2 ml/min) passed on the column Afterwards the column is washed with deionized water using a flow rate of 5 ml/min. Eluation of tiotropiumbicarbonate 2 from the column was detected using a UV-reader working at 240 nm. The fraction containing tiotropiumbicarbonate was collected (approx. 150 ml), the concentration was approx. 3 mg/ml, the pH was around 6.5.

The tiotropium bicarbonate 2 is preferably stored in form of this solution. In the alternative 2 is freshly prepared in advance to every salt form preparation. This procedure is usually applied in the following examples.

### Example 1

### Crystalline Anhydrous Tiotropium Benzenesulphonate

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of benzenesulfonic acid in 10 ml of water. The resulting solution was stored at 4° C. in the refrigerator overnight. The next day crystals of the besylate of tiotropium were formed. These were filtered, washed with cold demineralised water and dried under vacuum. Melting point: 237±3° C. (DSC);

the crystal structure of this salt was solved by single crystal X-ray diffraction analysis (see table 1).

TABLE 1

| Empirical formula | C₁₉H₂₂NO₄S₂⁺•C₆H₅SO₃⁻ |
|---|---|
| Formula weight | 549.66 |
| T [K] | 293(2) |
| λ [Å] | 0.71073 |
| Crystal system | Orthorhombic |
| Space group | Pbca |
| Unit cell dimensions | |
| a [Å] | 10.6460(7) |
| b [Å] | 12.8410(10) |
| c [Å] | 36.605(3) |
| V [Å³] | 5004.1(6) |
| Z | 8 |
| Dₘ [g/cm³] | 1.459 |
| F (000) | 2304 |
| Crystal size [mm³] | 0.7 × 0.4 × 0.1 |
| θ range | 3 to 27°. |
| Reflections collected | 10619 |
| Independent reflections | 5640 [Rᵢₙₜ = 0.0284] |
| Data/restraints/parameters | 5640/0/458 |
| S | 1.024 |
| R [I > 2σ(I)] | R1 = 0.0557, wR2 = 0.1398 |
| R indices (all data) | R1 = 0.0766, wR2 = 0.1523 |
| Extinction coefficient | 0.0023(7) |

### Example 2

### Crystalline Anhydrous Tiotropium Trifluoromethanesulphonate

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of trifluormethanesulfonic acid in 10 ml of water. The resulting solution was stored at 4° C. in the refrigerator overnight. The next day crystals of the triflate of tiotropium were formed. These were filtered, washed with cold demineralised water and dried under vacuum.

Melting point: 188±3° C. (DSC); the crystal structure of this salt was solved by single crystal X-ray diffraction analysis (see table 2).

TABLE 2

| Empirical formula | C₁₉H₂₂NO₄S₂⁺•CF₃SO₃⁻ |
|---|---|
| Fw | 541.57 |
| T [K] | 293(2) K |
| λ [Å] | 0.71073 Å |
| Crystal system | Monoclinic |
| Space group | P 2₁/c |
| Unit cell dimensions | |
| a [Å] | 12.4500(7) |
| b [Å] | 13.1090(9) |
| c [Å] | 17.9840(14) |
| β [°] | 129.176(2) |
| V [Å³] | 2275.3(3) |
| Z | 4 |
| Dₘ [g/cm³] | 1.581 |
| F (000) | 1120 |
| Crystal size [mm³] | 0.6 × 0.5 × 0.4 |
| θ range [°] | 2 → 27.5. |
| Reflections collected | 15539 |
| Independent reflections | 5198 [Rᵢₙₜ = 0.0353] |
| Data/restraints/parameters | 5198/0/411 |
| S | 1.021 |
| R [I > 2σ(I)] | R1 = 0.0466, wR2 = 0.1143 |
| R indices (all data) | R1 = 0.0641, wR2 = 0.1269 |
| Extinction coefficient | 0.028(2) |

### Example 3

### Crystalline Tiotropium Salicylate Monohydrate (Form I)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of salicylic acid in 10 ml of water. From the resulting solution water was evaporated under vacuum over a water bath which was kept below 35° C. The obtained solid was further dried under vacuum yielding a hydrated form of the salicylate of tiotropium. The crystal structure of this salt was solved by single crystal X-ray diffraction analysis (see table 3).

TABLE 3

| | |
|---|---|
| Empirical formula | C₁₉H₂₂NO₄S₂⁺•C₇H₅O₃⁻•H₂O |
| Fw | 547.62 |
| T [K] | 293(2) K |
| λ [Å] | 0.71073 Å |
| Crystal system | Triclinic |
| Space group | P −1 |
| Unit cell dimensions | |
| a [Å] | 10.8380(3) |
| b [Å] | 10.8610(3) |
| c [Å] | 12.2310(4) |
| α [°] | 76.199(2) |
| β [°] | 71.878(2) |
| γ [°] | 74.220(2) |
| V [Å³] | 1297.95(7) |
| Z | 2 |
| Dₘ [g/cm³] | 1.401 |
| F (000) | 576 |
| Crystal size [mm³] | 0.6 × 0.4 × 0.2 |
| θ range [°] | 3 to 27.5. |
| Reflections collected | 6946 |
| Independent reflections | 5568 [Rᵢₙₜ = 0.0286] |
| Data/restraints/parameters | 5568/0/450 |
| S | 1.023 |
| R [I > 2σ(I)] | R1 = 0.0507, wR2 = 0.1208 |
| R indices (all data) | R1 = 0.0744, wR2 = 0.1365 |

### Example 4

### Crystalline Anhydrous Tiotropium Salicylate (Form II)

1.00 g of tiotropium bromide monohydrate (according to WO 02/30928) was dissolved in in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of salicylic acid in 10 ml of water. From the resulting solution water was evaporated under vacuum over a water bath which was kept below 35° C. The obtained solid was further dried under vacuum yielding crystalline material of the salicylate of tiotropium. Approx. 600 mg of the crystalline tiotropium salicylate are dissolved in 4 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a mixture of 1-butanol/methanol=50:50 was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 25° C. At this temperature the plate remained for a hold time of 24 h. The plates are opened afterwards the solid is obtained by evaporation of the solvent at room temperature in a vacuum chamber (13 kPa). Melting point: 195±3° C. (DSC); table 4a summarizes the X-ray powder reflections obtained for this form.

TABLE 4a

| 2Θ [°] | d [Å] | I/Iₒ [%] |
|---|---|---|
| 6.54 | 13.50 | 56 |
| 9.42 | 9.38 | 11 |
| 10.54 | 8.38 | 11 |
| 12.22 | 7.23 | 79 |
| 12.86 | 6.88 | 7 |
| 13.86 | 6.38 | 16 |
| 16.22 | 5.46 | 100 |
| 16.82 | 5.26 | 42 |
| 18.38 | 4.82 | 23 |
| 19.54 | 4.54 | 31 |
| 20.62 | 4.30 | 97 |
| 21.98 | 4.04 | 23 |
| 22.98 | 3.87 | 9 |
| 23.42 | 3.79 | 14 |
| 24.38 | 3.65 | 8 |
| 25.06 | 3.55 | 18 |
| 26.06 | 3.42 | 27 |
| 27.38 | 3.25 | 21 |
| 28.10 | 3.18 | 6 |
| 29.34 | 3.06 | 10 |
| 29.82 | 3.02 | 7 |

The crystal structure of this salt was also solved by single crystal X-ray diffraction analysis (see table 4b).

TABLE 4b

| Empirical formula | C₁₉H₂₂NO₄S₂⁺•C₇H₅O₃⁻ |
|---|---|
| Formula weight | 529.61 |
| Temperature | 293(2) |
| Wavelength | 0.71073 |
| Crystal system | Monoclinic |
| Space group | C 2/c |
| Unit cell dimensions | |
| a [Å] | 13.273(2) |
| b [Å] | 13.865(2) |
| c [Å] | 28.042(4) |
| β [°] | 101.98(2) |
| V [Å³] | 5048.1(10) |
| Z | 8 |
| Density (calculated) | 1.394 |
| F (000) | 2224 |
| Crystal size | 0.3 × 0.2 × 0.2 |
| Theta range for data collection | 1.5 → 25 |
| Reflections collected | 7741 |
| Independent reflections | 4037 [Rᵢₙₜ = 0.0526] |
| Data/restraints/parameters | 4037/0/335 |
| Goodness-of-fit on F² | 1.090 |
| Final R indices [I > 2sigma (I)] | R1 = 0.0842, wR2 = 0.2068 |
| R indices (all data) | R1 = 0.1453, wR2 = 0.2482 |
| Extinction coefficient | 0.0046(8) |

### Example 5

### Crystalline Anhydrous Tiotropium Salicylate (Form III)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of salicylic acid in 10 ml of water. From the resulting solution water was evaporated under vacuum over a water bath which was kept below 35° C. The obtained solid was further dried under vacuum yielding crystalline material of the salicylate of tiotropium.

Approx. 600 mg of the crystalline tiotropium salicylate are dissolved in 4 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of N,N-dimethyl-acetamide (=DMA) was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 5° C. At this temperature the plate remained for a hold time of 24 h. The plates are opened afterwards the solid is obtained by evaporation of the solvent at room temperature in a vacuum chamber (13 kPa). Table 5 summarizes the X-ray powder reflections obtained for this form.

TABLE 5

| 2Θ [°] | d [Å] | I/Iₒ [%] |
|---|---|---|
| 8.18 | 10.80 | 51 |
| 10.86 | 8.14 | 16 |
| 11.22 | 7.88 | 22 |
| 12.98 | 6.81 | 15 |
| 13.62 | 6.49 | 52 |
| 14.18 | 6.24 | 15 |
| 15.74 | 5.62 | 17 |
| 16.34 | 5.42 | 83 |
| 17.70 | 5.00 | 37 |
| 18.94 | 4.68 | 34 |
| 20.74 | 4.28 | 58 |
| 21.82 | 4.07 | 15 |
| 22.58 | 3.93 | 100 |
| 23.42 | 3.79 | 27 |
| 23.98 | 3.71 | 18 |
| 24.98 | 3.56 | 33 |
| 26.02 | 3.42 | 16 |
| 27.78 | 3.21 | 7 |
| 28.66 | 3.11 | 12 |

### Example 6

### Crystalline Tiotropium Salicylate Monohydrate (Form IV)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of salicylic acid in 10 ml of water. From the resulting solution water was evaporated under vacuum over a water bath which was kept below 35° C. The obtained solid was further dried under vacuum yielding crystalline material of the salicylate of tiotropium. Approx. 600 mg of the crystalline tiotropium salicylate are dissolved in 4 ml of a mixture of acetone/water=80:20. The solvent was gently evaporated at room temperature in a vacuum chamber (13 kPa). The crystalline material obtained was tiotropium salicylate, form IV which is another hydrated form (monohydrate II) of tiotropium salicylate. Melting point of 193±3° C. (DSC);

table 6 summarizes the X-ray powder reflections obtained for this form.

TABLE 6

| 2 Θ [°] | d [Å] | I/Iₒ [%] |
|---|---|---|
| 7.38 | 11.98 | 22 |
| 8.74 | 10.12 | 84 |
| 10.86 | 8.15 | 8 |
| 12.06 | 7.34 | 20 |
| 13.14 | 6.74 | 5 |
| 13.66 | 6.48 | 38 |
| 14.14 | 6.26 | 13 |
| 14.58 | 6.07 | 27 |
| 15.58 | 5.69 | 6 |
| 16.82 | 5.27 | 17 |
| 17.54 | 5.06 | 83 |
| 18.06 | 4.91 | 53 |
| 19.02 | 4.67 | 23 |
| 20.02 | 4.43 | 11 |
| 20.66 | 4.30 | 3 |
| 21.86 | 4.07 | 100 |
| 22.70 | 3.92 | 47 |
| 23.18 | 3.84 | 22 |
| 23.58 | 3.77 | 31 |
| 24.18 | 3.68 | 11 |
| 24.42 | 3.64 | 10 |
| 25.26 | 3.53 | 32 |
| 26.66 | 3.34 | 7 |
| 27.78 | 3.21 | 15 |
| 28.82 | 3.10 | 4 |
| 29.18 | 3.06 | 11 |
| 29.62 | 3.02 | 5 |

### Example 7

### Crystalline Tiotropium Hydrogenesulphate Monohydrate (Form I)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of sulphuric acid in 10 ml of water. The resulting solution was stored at 4° C. in the refrigerator overnight. The next day crystals of a hydrated form of the hydrogenesulphate of tiotropium were formed. These were filtered, washed with cold demineralised water and dried under vacuum. The crystal structure of this salt was solved by single crystal X-ray diffraction analysis (see table 7).

TABLE 7

| Empirical formula | C₁₉H₂₂NO₄S₂⁺•HSO₄•H₂O |
|---|---|
| Fw | 507.58 |
| T [K] | 293(2)K |
| λ [Å] | 0.71073 |
| Crystal system | Triclinic |
| Space group | P-1 |
| Unit cell dimensions | |
| a [Å] | 9.3750(2) |
| b [Å] | 11.6470(2) |
| c [Å] | 20.5450(5) |
| α [°] | 91.6260(9) |
| β [°] | 95.7210(9) |
| γ [°] | 91.8520(12) |
| V [Å³] | 2229.85(8) |
| Z | 4 |
| Dₘ [g/cm³] | 1.509 |
| F(000) | 1060 |
| Crystal size [mm³] | 0.4 × 0.4 × 0.1 |
| θ range [°] | 2 to 27.5. |
| Reflections collected | 15298 |
| Independent reflections | 10156 [Rᵢₙₜ = 0.0317] |
| Data/restraints/parameters | 10156/0/624 |
| S | 1.016 |
| R [I > 2σ(I)] | R1 = 0.0699, wR2 = 0.1744 |
| R indices (all data) | R1 = 0.1278, wR2 = 0.2086 |
| Extinction coefficient | 0.0010(9) |

### Example 8

### Crystalline Anhydrous Tiotropium Hydrogenesulphate (Form II)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of sulphuric acid in 10 ml of water. The resulting solution was stored at 4° C. in the refrigerator overnight. The next day crystals of a hydrated form of the hydrogenesulphate of tiotropium were formed. These were filtered, washed with cold demineralised water and dried under vacuum.

Approx. 900 mg of the crystalline tiotropium hydrogenesulphate are dissolved in 2 ml of water. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a mixture of 1-butanol/methanol=50:50 was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 25° C. At this temperature the plate remained for a hold time of 24 h. The plates are opened afterwards the solid is obtained by evaporation of the solvent at room temperature in a vacuum chamber (13 kPa). Melting point: 188±3° C. (DSC); table 8a summarizes the X-ray powder reflections obtained for this form.

TABLE 8a

| 2 Θ [°] | d [Å] | I/Iₒ [%] |
|---|---|---|
| 5.38 | 16.41 | 21 |
| 6.18 | 14.28 | 10 |
| 7.94 | 11.12 | 14 |
| 11.46 | 7.71 | 40 |
| 12.30 | 7.19 | 27 |
| 13.62 | 6.49 | 21 |
| 15.62 | 5.67 | 79 |
| 16.50 | 5.37 | 38 |
| 17.06 | 5.21 | 17 |
| 17.66 | 5.02 | 11 |
| 18.50 | 4.79 | 66 |
| 19.06 | 4.65 | 54 |
| 19.62 | 4.52 | 44 |
| 20.30 | 4.37 | 36 |
| 20.78 | 4.27 | 39 |
| 21.62 | 4.11 | 46 |
| 22.46 | 3.95 | 100 |
| 23.06 | 3.85 | 33 |
| 24.26 | 3.66 | 17 |
| 24.94 | 3.57 | 26 |
| 25.50 | 3.49 | 19 |
| 26.10 | 3.41 | 17 |
| 27.30 | 3.26 | 31 |
| 28.34 | 3.15 | 13 |
| 29.18 | 3.06 | 20 |

The crystal structure of this salt was also solved by single crystal X-ray diffraction analysis (see table 8b).

TABLE 8b

| | |
|---|---|
| Empirical formula | C₁₉H₂₂NO₄S₂•HSO₄⁻ |
| Formula weight | 489.56 |
| Temperature | 293(2) K |
| Wavelength | 0.71073 Å |
| Crystal system | Monoclinic |
| Space group | P 2₁/c |
| Unit cell dimensions | |
| a [Å] | 8.0390(2) Å |
| b [Å] | 15.989(1) Å |
| c [Å] | 33.190(2) Å |
| β [°] | 90.265(2) |
| Volume [Å³] | 4266.0(2) |
| Z | 8 |
| Density (calculated) | 1.524 |
| Absorption coefficient | 0.395 |
| F(000) | 2048 |
| Crystal size | 0.3 × 0.3 × 0.2 |
| Theta range for data collection | 1.5 → 24 |
| Reflections collected | 14141 |
| Independent reflections | 6383 [Rᵢₙₜ = 0.0713] |
| Data/restraints/parameters | 6383/0/563 |
| Goodness-of-fit on F² | 1.130 |
| Final R indices [I > 2sigma(I)] | R1 = 0.1396, wR2 = 0.3623 |
| R indices (all data) | R1 = 0.1561, wR2 = 0.3773 |

### Example 9

### Crystalline Anhydrous Tiotropium Hydrogenesulphate (Form III)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of sulphuric acid in 10 ml of water. The resulting solution was stored at 4° C. in the refrigerator overnight. The next day crystals of a hydrated form of the hydrogenesulphate of tiotropium were formed. These were filtered, washed with cold demineralised water and dried under vacuum.

Approx. 900 mg of the crystalline tiotropium hydrogenesulphate are dissolved in 2 ml of water. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a acetone was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 25° C. At this temperature the plate remained for a hold time of 24 h. The plates are opened afterwards the solid is obtained by evaporation of the solvent at room temperature in a vacuum chamber (13 kPa). Melting point: 185±5° C. (DSC); table 9 summarizes the X-ray powder reflections obtained for this form.

TABLE 9

| 2 Θ [°] | d [Å] | I/Iₒ [%] |
|---|---|---|
| 5.42 | 16.29 | 66 |
| 7.66 | 11.53 | 9 |
| 10.78 | 8.20 | 16 |
| 11.42 | 7.74 | 15 |
| 12.06 | 7.33 | 40 |
| 12.54 | 7.05 | 28 |
| 13.58 | 6.51 | 11 |
| 14.06 | 6.29 | 22 |
| 15.50 | 5.71 | 53 |
| 15.94 | 5.55 | 53 |
| 16.94 | 5.23 | 55 |
| 18.26 | 4.85 | 52 |
| 19.78 | 4.48 | 73 |
| 20.38 | 4.35 | 86 |
| 21.58 | 4.11 | 33 |
| 22.82 | 3.89 | 100 |
| 23.74 | 3.74 | 10 |
| 24.46 | 3.63 | 15 |
| 25.94 | 3.43 | 13 |
| 26.70 | 3.33 | 11 |
| 27.34 | 3.26 | 14 |
| 29.10 | 3.06 | 11 |
| 29.66 | 3.01 | 20 |

### Example 10

### Crystalline Tiotropium Dihydrogenephosphate Monohydrate

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of phosphoric acid in 10 ml of water. The resulting solution was stored at 4° C. in the refrigerator overnight. The next day crystals of a hydrated form of the dihydrogenephosphate of tiotropium were formed. These were filtered, washed with cold demineralised water and dried under vacuum.

Melting point: 222±3° C. (DSC); The crystal structure of this salt was solved by single crystal X-ray diffraction analysis (see table 10a).

TABLE 10a

| Empirical formula | C₁₉H₂₂NO₄S₂⁺•H₂PO₄⁻•H₂O |
|---|---|
| Fw | 507.5 |
| T [K] | 293(2) |
| λ [Å] | 0.71073 |
| Crystal system | Monoclinic |
| Space group | P 2₁/c |
| Unit cell dimensions | |
| a [Å] | 22.6740(17) Å |
| b [Å] | 6.6690(9) Å |
| c [Å] | 15.061(3) Å |
| β [°] | 96.476(8)°. |
| V [Å³] | 2262.9(6) Å³ |
| Z | 4 |
| Dₘ [g/cm³] | 1.490 |
| F(000) | 1064 |
| Crystal size [mm³] | 0.2 × 0.1 × 0.5 |
| θ range [°] | 2 → 22.5. |
| Reflections collected | 4189 |
| Independent reflections | 2509 [Rᵢₙₜ = 0.1336] |
| Data/restraints/parameters | 2509/0/290 |
| S | 1.039 |
| R [I > 2σ(I)] | R1 = 0.0842, wR2 = 0.1859 |
| R indices (all data) | R1 = 0.1632, wR2 = 0.2344 |
| Extinction coefficient | 0.07(2) |

Table 10b summarizes the X-ray powder reflections obtained for this form.

TABLE 10b

| 2 Θ [°] | d [Å] | I/Iₒ [%] |
|---|---|---|
| 4.02 | 21.97 | 44 |
| 7.98 | 11.07 | 15 |
| 12.02 | 7.35 | 78 |
| 13.58 | 6.51 | 56 |
| 14.14 | 6.26 | 81 |
| 15.62 | 5.67 | 59 |
| 16.50 | 5.37 | 30 |
| 17.14 | 5.17 | 22 |
| 18.10 | 4.90 | 97 |
| 18.58 | 4.77 | 64 |
| 19.22 | 4.61 | 67 |
| 20.38 | 4.35 | 40 |
| 21.02 | 4.22 | 100 |
| 22.50 | 3.95 | 58 |
| 23.22 | 3.83 | 31 |
| 24.46 | 3.63 | 76 |
| 25.50 | 3.49 | 20 |
| 26.38 | 3.37 | 19 |
| 27.54 | 3.23 | 38 |
| 28.06 | 3.19 | 20 |
| 28.94 | 3.08 | 16 |
| 29.86 | 2.99 | 18 |

### Example 11

### Crystalline Ditiotropium Ethanedisulphonate Hydrate

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of ethanedisulphonic acid in 10 ml of water. From the resulting solution water was evaporated under vacuum over a water bath which was kept below 35° C. The obtained solid was further dried under vacuum yielding a hydrated form of the ethanedisulphonate of tiotropium.

The crystal structure of this salt was solved by single crystal X-ray diffraction analysis (see table 11).

TABLE 11

| Empirical formula | 2•(C₁₉H₂₂NO₄S₂⁺)•C₂H₄O₆S₂²⁻•10H₂O |
|---|---|
| Fw | 1152.32 |
| T [K] | 263(2) K |
| λ [Å] | 0.71073 Å |
| Crystal system | Triclinic |
| Space group | P-1 |
| Unit cell dimensions | |
| a [Å] | 9.2700(8) |
| b [Å] | 12.8920(13) |
| c [Å] | 22.579(2) |
| α [°] | 103.876(3) |
| β [°] | 93.620(4) |
| γ [°] | 90.327(5) |
| V [Å³] | 2613.8(4) |
| Z | 2 |
| Dₘ [g/cm³] | 1.464 Mg/m³ |
| F(000) | 1218 |
| Crystal size [mm³] | 0.4 × 0.2 × 0.2 mm³ |
| θ range [°] | 1.5 to 26.5 |
| Reflections collected | 3753 |
| Independent reflections | 3360 [Rᵢₙₜ = 0.0372] |
| Data/restraints/parameters | 3360/0/292 |
| S | 2.605 |
| R [I > 2σ(I)] | R1 = 0.1919, wR2 = 0.4322 |
| R indices (all data) | R1 = 0.2293, wR2 = 0.4495 |
| Extinction coefficient | 0.026(6) |

### Example 12

### Crystalline Tiotropium Xinafoate Monohydrate (Form I)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of 1-hydroxy-2-naphthoic acid in 10 ml of water. From the resulting solution water was evaporated under vacuum over a water bath which was kept below 35° C. The obtained solid was further dried under vacuum yielding a hydrated form of the xinafoate of tiotropium. Melting point: 170±3° C. (DSC); the crystal structure of this salt was solved by single crystal X-ray diffraction analysis analysis (see table 12a).

TABLE 12a

| Empirical formula | C₁₉H₂₂NO₄S₂⁺•C₁₁H₇O₃⁻•H2O |
|---|---|
| Fw | 597.68 |
| T [K] | 293(2) |
| λ [Å] | 0.71073 |
| Crystal system | Monoclinic |
| Space group | P 2₁/c |
| Unit cell dimensions | |
| a [Å] | 13.5460(16) |
| b [Å] | 16.491(3) |
| c [Å] | 13.263(2) |
| β [°] | 100.51(2) |
| V [Å³] | 2913.0(7) |
| Z | 4 |
| Dₘ [g/cm³] | 1.363 |
| F(000) | 1256 |
| Crystal size [mm³] | 0.3 × 0.3 × 0.2 |
| θ range [°] | 2.5 → 27.5 |
| Reflections collected | 19684 |
| Independent reflections | 6685 [Rᵢₙₜ = 0.0682] |
| Data/restraints/parameters | 6685/0/406 |
| S | 1.007 |
| R [I > 2σ(I)] | R1 = 0.0586, wR2 = 0.1255 |
| R indices (all data) | R1 = 0.1287, wR2 = 0.1566 |
| Extinction coefficient | 0.0105(12) |

Table 12b summarizes the X-ray powder reflections obtained for this form.

TABLE 12b

| 2 Θ [°] | d [Å] | I/Iₒ [%] |
|---|---|---|
| 6.70 | 13.18 | 25 |
| 8.62 | 10.25 | 27 |
| 10.14 | 8.71 | 9 |
| 10.74 | 8.23 | 20 |
| 11.66 | 7.59 | 7 |
| 13.38 | 6.61 | 16 |
| 14.02 | 6.31 | 34 |
| 14.90 | 5.94 | 63 |
| 16.22 | 5.48 | 8 |
| 17.06 | 5.19 | 18 |
| 18.14 | 4.88 | 17 |
| 19.22 | 4.61 | 14 |
| 20.02 | 4.43 | 8 |
| 20.78 | 4.27 | 14 |
| 21.54 | 4.12 | 100 |
| 22.10 | 4.02 | 9 |
| 22.90 | 3.88 | 28 |
| 23.70 | 3.79 | 7 |
| 24.82 | 3.58 | 15 |
| 26.30 | 3.38 | 13 |
| 27.10 | 3.29 | 11 |
| 27.90 | 3.24 | 10 |
| 28.34 | 3.19 | 9 |
| 28.94 | 3.13 | 5 |
| 29.42 | 3.08 | 3 |

### Example 13

### Crystalline Anhydrous Tiotropium Xinafoate (Form II)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of 1-hydroxy-2-naphthoic acid in 10 ml of water. From the resulting solution water was evaporated under vacuum over a water bath which was kept below 35° C. The obtained solid was further dried under vacuum yielding a powdery material of the xinafoate of tiotropium. Approx. 500 mg of the this tiotropium xinafoate are dissolved in 4 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a mixture of diisopropyl ether/methanol=50:50 was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 25° C. At this temperature the plate remained for a hold time of 24 h. The plates are opened afterwards the solid is obtained by evaporation of the solvent at room temperature in a vacuum chamber (13 kPa). Melting point by DSC: 180±3° C.; the crystal structure of this salt was solved by single crystal X-ray diffraction analysis (see table 13a).

TABLE 13a

| | |
|---|---|
| Empirical formula | C₁₉H₂₂NO₇S₂⁺•C₁₁H₇O₃⁻ |
| Formula weight | 579.66 |
| Temperature | 293(2) |
| Wavelength | 0.71073 |
| Crystal system | Monoclinic |
| Space group | P 2₁/c |
| Unit cell dimensions | |
| a [Å] | 15.9650(4) |
| b [Å] | 13.2330(3) |
| c [Å] | 14.1810(5) |
| β [°] | 111.781(2) |
| Volume [Å³] | 2782.06(14) |
| Z | 4 |
| Density (calculated) | 1.384 |
| F(000) | 1216 |
| Crystal size | 0.45 × 0.3 × 0.2 |
| Theta range for data collection | 3 → 26 |
| Reflections collected | 14803 |
| Independent reflections | 5437 [Rᵢₙₜ = 0.0318] |
| Data/restraints/parameters | 5437/0/411 |
| Goodness-of-fit on F² | 1.043 |
| Final R indices [I > 2sigma(I)] | R1 = 0.0631, wR2 = 0.1766 |
| R indices (all data) | R1 = 0.0821, wR2 = 0.1935 |

Table 13b summarizes the X-ray powder reflections obtained for this form.

TABLE 13b

| 2 Θ [°] | d [Å] | I/Iₒ [%] |
|---|---|---|
| 6.06 | 14.57 | 62 |
| 9.86 | 8.96 | 20 |
| 12.58 | 7.03 | 14 |
| 13.50 | 6.55 | 100 |
| 14.78 | 5.99 | 56 |
| 15.90 | 5.57 | 30 |
| 17.22 | 5.14 | 25 |
| 18.06 | 4.91 | 69 |
| 19.30 | 4.59 | 36 |
| 20.54 | 4.32 | 12 |
| 21.46 | 4.14 | 32 |
| 22.66 | 3.92 | 64 |
| 23.30 | 3.81 | 16 |
| 23.98 | 3.79 | 5 |
| 25.50 | 3.49 | 37 |
| 26.46 | 3.36 | 13 |
| 27.18 | 3.28 | 27 |
| 28.26 | 3.15 | 11 |
| 28.90 | 3.12 | 7 |
| 29.70 | 3.00 | 10 |

### Example 14

### Crystalline Tiotropium Xinafoate Monohydrate (Form III)

6.00 g of tiotropium bromide monohydrate was dissolved in 350 ml of demineralised water at 25° C. 150 ml of bicarbonate loaded IER slurry was added to this solution and stirred for 5 minutes. A silver nitrate test as described above indicated the completion of the exchange. After stirring for an additional 5 minutes the slurry was filtered with PTFE filters with a pore size of 10 μm and washed with 150 ml water. 1.1 equivalents of 1-hydroxy-2-naphthoic acid in 70 ml isopropanol were added to the filtrate. From the resulting solution water and isopropanol was evaporated under gentle conditions (40-50° C.). To the obtained oil first 50 ml isopropanol and than 40 ml ethanol was added and heated up to 70° C. A clear slightly brownish solution was obtained which was filtered with charcoal. The obtained clear solution was stirred at room temperature. After 72 hours stirring at room temperature a suspension was obtained. the crystals were filtered off, washed with a small amount of cold isopropanol and than dried over night at room temperature. Melting point by DSC: 110±3° C. (under dehydration); the crystal structure of this salt was solved by single crystal X-ray diffraction analysis. (see table 14a).

TABLE 14a

| | |
|---|---|
| Empirical formula | C₁₉H₂₂NO₄S₂⁺•C₁₁H₇O₃⁻•H₂O |
| Formula weight | 597.68 |
| Temperature | 293(2) |
| Wavelength | 0.71073 |
| Crystal system | Monoclinic |
| Space group | P 2₁/c |
| Unit cell dimensions | |
| a [Å] | 13.2470(6) |
| b [Å] | 11.3590(6) |
| c [Å] | 20.9500(7) |
| β [°] | 118.229(4) |
| Volume [Å³] | 2777.5(2) |
| Z | 4 |
| Density (calculated) | 1.429 |
| F(000) | 1256 |
| Crystal size | 0.45 × 0.3 × 0.25 |
| Theta range for data collection | 2 → 25. |
| Reflections collected | 3999 |
| Independent reflections | 3092 [Rᵢₙₜ = 0.0568] |
| Data/restraints/parameters | 3092/0/375 |
| Goodness-of-fit on F² | 1.889 |
| Final R indices [I > 2sigma(I)] | R1 = 0.1001, wR2 = 0.2694 |
| R indices (all data) | R1 = 0.1208, wR2 = 0.2837 |

Table 14b summarizes the X-ray powder reflections obtained for this form.

TABLE 14b

| 2 Θ [°] | d [Å] | I/Iₒ [%] |
|---|---|---|
| 8.93 | 9.90 | 73 |
| 9.13 | 9.68 | 10 |
| 9.55 | 9.26 | 5 |
| 10.32 | 8.57 | 8 |
| 10.84 | 8.16 | 45 |
| 11.84 | 7.47 | 11 |
| 12.32 | 7.18 | 26 |
| 13.23 | 6.69 | 16 |
| 13.56 | 6.53 | 4 |
| 14.79 | 5.99 | 49 |
| 15.16 | 5.84 | 13 |
| 15.59 | 5.68 | 19 |
| 16.31 | 5.43 | 37 |
| 17.04 | 5.20 | 86 |
| 17.94 | 4.94 | 3 |
| 18.28 | 4.85 | 14 |
| 18.64 | 4.76 | 27 |
| 19.15 | 4.63 | 16 |
| 19.55 | 4.54 | 85 |
| 20.09 | 4.42 | 100 |
| 20.70 | 4.29 | 45 |
| 21.49 | 4.13 | 13 |
| 21.81 | 4.07 | 37 |
| 22.84 | 3.89 | 17 |
| 23.08 | 3.85 | 12 |
| 23.84 | 3.73 | 10 |
| 24.42 | 3.65 | 3 |
| 24.78 | 3.59 | 51 |
| 25.07 | 3.55 | 12 |
| 25.32 | 3.52 | 13 |
| 26.62 | 3.35 | 23 |
| 27.28 | 3.27 | 30 |
| 27.72 | 3.22 | 5 |
| 28.02 | 3.18 | 10 |
| 28.75 | 3.10 | 20 |
| 29.61 | 3.02 | 10 |
| 29.93 | 2.98 | 4 |

### Example 15

### Crystalline Tiotropium Fumarate Ethanolate (Form I)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of fumaric acid in 10 ml of water. From the resulting solution water was evaporated under vacuum over a water bath which was kept below 35° C. The obtained solid was further dried under vacuum yielding an amorphous oil of the fumarate of tiotropium. This material was recrystallised afterwards from ethanol as followed. About 100 mg of the salt was suspended in absolute ethanol. The mixture was heated to 50° C. and kept at that temperature for approximately 1h until complete dissolution was observed. The solution was not filtered and cooled slowly to room temperature. After several days, crystals of a solvated form containing ethanol of the fumarate of tiotropium were formed, which were isolated by filtration.

The crystal structure of this salt was solved by single crystal X-ray diffraction analysis (see table 15).

TABLE 15

| Empirical formula | C₁₉H₂₂NO₄S₂⁺•C₄H₃O₄⁻•C₂H₆O |
|---|---|
| Fw | 553.63 |
| T [K] | 293(2) |
| λ [Å] | 0.71073 |
| Crystal system | Orthorhombic |
| Space group | P bca |
| Unit cell dimensions | |
| a [Å] | 15.3830(7) |
| b [Å] | 16.8490(7) |
| c [Å] | 20.0900(12) |
| V [Å³] | 5207.1(4) |
| Z | 8 |
| Dₘ [g/cm³] | 1.412 |
| F(000) | 2336 |
| Crystal size [mm³] | 0.2 × 0.2 × 0.08 |
| θ range [°] | 3 → 22.5. |
| Reflections collected | 6183 |
| Independent reflections | 3285 [Rᵢₙₜ = 0.0458] |
| Data/restraints/parameters | 3285/0/336 |
| S | 1.040 |
| R [I > 2σ(I)] | R1 = 0.0923, wR2 = 0.2229 |
| R indices (all data) | R1 = 0.1243, wR2 = 0.2489 |
| Extinction coefficient | 0.0020(9) |

### Example 16

### Crystalline Anhydrous Tiotropium Fumarate (Form II)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of fumaric acid in 10 ml of water. From the resulting solution water was evaporated under vacuum over a water bath which was kept below 35° C. The obtained solid was further dried under vacuum yielding an amorphous oil of the fumarate of tiotropium. This material was recrystallised afterwards from ethanol as followed. About 100 mg of the salt was suspended in absolute ethanol. The mixture was heated to 50° C. and kept at that temperature for approximately 1h until complete dissolution was observed. The solution was not filtered and cooled slowly to room temperature. After several weeks, large crystals of an anhydrous fumarate of tiotropium were formed, which were isolated by filtration. Melting point: 180±3° C. (DSC);

The crystal structure of this salt was solved by single crystal X-ray diffraction analysis (see table 16a).

TABLE 16a

| | |
|---|---|
| Empirical formula | C₁₉H₂₂NO₄S₂⁺•C₄H₃O₄⁻ |
| Fw | 507.56 |
| T [K] | 293(2) K |
| λ [Å] | 0.71073 Å |
| Crystal system | Triclinic |
| Space group | P-1 |
| Unit cell dimensions | |
| a [Å] | 7.4980(3) |
| b [Å] | 9.4900(4) |
| c [Å] | 17.0110(7) |
| α [°] | 102.125(2) |
| β [°] | 96.182(2) |
| γ [°] | 99.289(2) |
| V [Å³] | 1155.27(8) |
| Z | 2 |
| Dₘ [g/cm³] | 1.459 |
| F(000) | 532 |
| Crystal size [mm³] | 0.45 × 0.4 × 0.2 |
| θ range [°] | 2.3 → 27.7 |
| Reflections collected | 6544 |
| Independent reflections | 4796 [Rᵢₙₜ = 0.0354] |
| Data/restraints/parameters | 4796/0/410 |
| S | 1.023 |
| R [I > 2σ(I)] | R1 = 0.0493, wR2 = 0.1201 |
| R indices (all data) | R1 = 0.0646, wR2 = 0.1322 |

Table 16b summarizes the X-ray powder reflections obtained for this form.

TABLE 16b

| 2Θ [°] | d [Å] | I/Iₒ [%] |
|---|---|---|
| 6.30 | 14.01 | 4 |
| 10.06 | 8.78 | 47 |
| 10.86 | 8.14 | 11 |
| 12.50 | 7.07 | 64 |
| 14.14 | 6.26 | 88 |
| 15.14 | 5.84 | 45 |
| 16.26 | 5.44 | 42 |
| 18.02 | 4.92 | 77 |
| 18.86 | 4.70 | 100 |
| 19.58 | 4.53 | 58 |
| 21.06 | 4.21 | 63 |
| 21.78 | 4.08 | 77 |
| 22.42 | 3.96 | 61 |
| 24.38 | 3.65 | 48 |
| 25.02 | 3.55 | 53 |
| 25.70 | 3.46 | 42 |
| 26.74 | 3.33 | 13 |
| 28.34 | 3.15 | 36 |
| 28.86 | 3.09 | 23 |
| 29.78 | 3.00 | 16 |

### Example 17

### Crystalline Anhydrous Tiotropium Fumarate (Form III)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of fumaric acid in 10 ml of water. From the resulting solution water was evaporated under vacuum over a water bath which was kept below 35° C. The obtained solid was further dried under vacuum yielding an amorphous oil of the fumarate of tiotropium.

Approx. 600 mg of the amorphous tiotropium fumarate are dissolved in 4 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of 1-methyl-2-pyrollidinone (=NMP, extra dry) was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 25° C. At this temperature the plate remained for a hold time of 24 h. The plates are opened afterwards the solid is obtained by evaporation of the solvent at room temperature in a vacuum chamber (13 kPa). Table 17 summarizes the X-ray powder reflections obtained for this form.

TABLE 17

| 2Θ [°] | d [Å] | I/Iₒ [%] |
|---|---|---|
| 9.02 | 9.79 | 21 |
| 10.02 | 8.82 | 35 |
| 10.82 | 8.17 | 9 |
| 12.42 | 7.12 | 51 |
| 13.46 | 6.57 | 11 |
| 14.06 | 6.29 | 65 |
| 15.14 | 5.84 | 46 |
| 16.22 | 5.46 | 48 |
| 16.82 | 5.26 | 37 |
| 17.94 | 4.94 | 69 |
| 18.82 | 4.71 | 68 |
| 19.54 | 4.54 | 100 |
| 21.02 | 4.22 | 49 |
| 21.66 | 4.10 | 68 |
| 22.38 | 3.97 | 46 |
| 23.74 | 3.74 | 32 |
| 24.26 | 3.66 | 46 |
| 24.82 | 3.58 | 50 |
| 25.66 | 3.47 | 44 |
| 26.22 | 3.39 | 29 |
| 28.26 | 3.15 | 25 |
| 28.94 | 3.08 | 24 |
| 29.70 | 3.00 | 17 |
| 30.34 | 2.94 | 22 |

### Example 18

### Crystalline Anhydrous Tiotropium Fumarate (Form IV)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of fumaric acid in to 10 ml of water. From the resulting solution water was evaporated under vacuum over a water bath which was kept below 35° C. The obtained solid was further dried under vacuum yielding an amorphous oil of the fumarate of tiotropium.

Approx. 600 mg of the amorphous tiotropium fumarate are dissolved in 4 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a mixture of acetonitrile/water=50:50 was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 5° C. At this temperature the plate remained for a hold time of 24 h. The plates are opened afterwards the solid is obtained by evaporation of the solvent at room temperature in a vacuum chamber (13 kPa). Melting point: 225±3° C. (DSC); table 18a summarizes the X-ray powder reflections obtained for this form.

TABLE 18a

| 2Θ [°] | d [Å] | I/Iₒ [%] |
|---|---|---|
| 8.18 | 10.80 | 10 |
| 10.22 | 8.65 | 18 |
| 11.10 | 7.96 | 84 |
| 12.70 | 6.96 | 12 |
| 13.38 | 6.61 | 53 |
| 15.22 | 5.81 | 34 |
| 16.54 | 5.35 | 64 |
| 17.58 | 5.04 | 35 |
| 18.46 | 4.80 | 100 |
| 19.10 | 4.64 | 76 |
| 20.54 | 4.32 | 35 |
| 21.42 | 4.14 | 50 |
| 21.94 | 4.05 | 58 |
| 22.42 | 3.96 | 77 |
| 23.86 | 3.73 | 6 |
| 24.58 | 3.63 | 4 |
| 25.42 | 3.50 | 15 |
| 26.14 | 3.40 | 18 |
| 26.82 | 3.32 | 20 |
| 27.30 | 3.26 | 15 |
| 27.70 | 3.22 | 46 |
| 28.14 | 3.17 | 20 |
| 28.86 | 3.09 | 15 |
| 29.62 | 3.01 | 20 |

A single crystal x-ray structure analysis of form IV of tiotropium fumarate showed that this form is in fact a co-crystal of a 2:1 salt (tiotropium: counter ion) of tiotropium fumarate which contains an additional mol of fumaric acid.

The crystal structure of this salt was solved by single crystal X-ray diffraction analysis (see table 18b).

TABLE 18b

| | |
|---|---|
| Empirical formula | 2C₁₉H₂₂NO₄S₂⁺•C₄H₂O₄²⁻•C₂H₄O₄ |
| Fw | 1015.12 |
| T [K] | 293(2) |
| λ [Å] | 0.71073 |
| Crystal system | Monoclinic |
| Space group | P 2₁/c |
| Unit cell dimensions | |
| a [Å] | 9.6910(2) |
| b [Å] | 14.5710(4) |
| c [Å] | 18.1580(4) |
| β [°] | 116.781(2) |
| V [Å³] | 2289.01(9) |
| Z | 2 |
| Dₘ [g/cm³] | 1.473 |
| F(000) | 1064 |
| Crystal size [mm³] | 0.4 × 0.4 × 0.2 |
| θ range [°] | 3 → 26 |
| Reflections collected | 11429 |
| Independent reflections | 4424 [Rᵢₙₜ = 0.0352] |
| Data/restraints/parameters | 4424/0/424 |
| S | 1.030 |
| R [I > 2σ(I)] | R1 = 0.0459, wR2 = 0.1070 |
| R indices (all data) | R1 = 0.0630, wR2 = 0.1178 |

### Example 19

### Crystalline Anhydrous Tiotropium Tartrate (Form I)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of D-tartaric acid in 10 ml of water. From the resulting solution water was evaporated under vacuum over a water bath which was kept below 35° C. The obtained solid was further dried under vacuum yielding an amorphous oil of the tartrate of tiotropium. Approx. 500 mg of the amorphous tiotropium tartrate are dissolved in 4 ml of a water. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a mixture of acetonitrile/water=50:50 was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 5° C. At this temperature the plate remained for a hold time of 24 h. The plates are opened afterwards the solid is obtained by evaporation of the solvent at room temperature in a vacuum chamber (13 kPa). Melting point: 180±3° C. (DSC); table 19 summarizes the X-ray powder reflections obtained for this form.

TABLE 19

| 2Θ [°] | d [Å] | I/Iₒ [%] |
|---|---|---|
| 4.38 | 20.16 | 100 |
| 8.74 | 10.11 | 24 |
| 10.78 | 8.20 | 27 |
| 12.18 | 7.25 | 15 |
| 12.82 | 6.90 | 38 |
| 13.90 | 6.36 | 6 |
| 14.46 | 6.12 | 16 |
| 16.22 | 5.46 | 58 |
| 17.42 | 5.08 | 66 |
| 17.98 | 4.93 | 38 |
| 18.86 | 4.70 | 14 |
| 20.10 | 4.41 | 99 |
| 21.58 | 4.11 | 20 |
| 22.14 | 4.02 | 11 |
| 23.66 | 3.76 | 7 |
| 24.38 | 3.65 | 11 |
| 25.90 | 3.44 | 4 |
| 26.46 | 3.36 | 9 |

### Example 20

### Crystalline Anhydrous Tiotropium Tartrate (Form II)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of D-tartaric acid in 10 ml of water. From the resulting solution water was evaporated under vacuum over a water bath which was kept below 35° C. The obtained solid was further dried under vacuum yielding an amorphous oil of the tartrate of tiotropium.

Approx. 500 mg of the amorphous tiotropium tartrate are dissolved in 4 ml of a water. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of ethanol was added to this vial. The whole 96 well plate is to sealed afterwards and heated up with a heating rate of 5° C./min to 50° C. at which the plate stays for an additional 30 minutes. Afterwards the plate is cooled with a cooling rate of 5° C./h to a final temperature of 25° C. At this temperature the plate remained for a hold time of 24 h. The plates are opened afterwards the solid is obtained by evaporation of the solvent at room temperature in a vacuum chamber (13 kPa). Melting point: 190+3° C. (DSC); table 20 summarizes the X-ray powder reflections obtained for this form.

TABLE 20

| 2Θ [°] | d [Å] | I/Iₒ [%] |
|---|---|---|
| 5.62 | 15.71 | 61 |
| 9.42 | 9.38 | 93 |
| 11.22 | 7.88 | 28 |
| 12.82 | 6.90 | 23 |
| 14.82 | 5.97 | 47 |
| 15.78 | 5.61 | 17 |
| 16.02 | 5.53 | 21 |
| 16.82 | 5.26 | 76 |
| 18.14 | 4.88 | 100 |
| 19.02 | 4.66 | 21 |
| 20.26 | 4.38 | 45 |
| 21.58 | 4.11 | 70 |
| 22.26 | 3.99 | 45 |
| 22.82 | 3.89 | 36 |
| 24.18 | 3.68 | 39 |
| 24.78 | 3.59 | 14 |
| 25.54 | 3.48 | 18 |
| 26.78 | 3.33 | 18 |
| 27.22 | 3.28 | 8 |
| 28.58 | 3.12 | 14 |
| 29.30 | 3.04 | 22 |
| 29.94 | 2.98 | 14 |

### Example 21

### Crystalline Anhydrous Tiotropium Tartrate (Form III)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of D-tartaric acid in 10 ml of water. From the resulting solution water was evaporated under vacuum over a water bath which was kept below 35° C. The obtained solid was further dried under vacuum yielding an amorphous oil of the tartrate of tiotropium.

Approx. 500 mg of the amorphous tiotropium tartrate are dissolved in 4 ml of a water. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a mixture of 1-methyl-2-pyrrolidinon (=NMP) was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 5° C. At this temperature the plate remained for a hold time of 24 h. The plates are opened afterwards the solid is obtained by evaporation of the solvent at room temperature in a vacuum chamber (13 kPa).

Table 21 summarizes the X-ray powder reflections obtained for this form.

TABLE 21

| 2Θ [°] | d [Å] | I/Iₒ [%] |
|---|---|---|
| 8.54 | 10.34 | 35 |
| 12.42 | 7.12 | 24 |
| 13.14 | 6.73 | 35 |
| 13.90 | 6.36 | 49 |
| 14.46 | 6.12 | 47 |
| 15.82 | 5.60 | 37 |
| 16.22 | 5.46 | 25 |
| 17.06 | 5.19 | 62 |
| 17.58 | 5.04 | 100 |
| 18.30 | 4.84 | 30 |
| 19.06 | 4.65 | 31 |
| 20.22 | 4.39 | 16 |
| 21.90 | 4.05 | 54 |
| 22.66 | 3.92 | 11 |
| 23.70 | 3.76 | 14 |
| 24.86 | 3.58 | 28 |
| 25.66 | 3.49 | 14 |
| 27.42 | 3.25 | 26 |
| 27.98 | 3.19 | 14 |
| 28.82 | 3.09 | 7 |
| 29.46 | 3.03 | 11 |

### Example 22

### Crystalline Anhydrous Tiotropium Succinate

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of succinic acid in to 10 ml of water. From the resulting solution water was evaporated under vacuum over a water bath which was kept below 35° C. The obtained solid was further dried under vacuum yielding an amorphous oil of the succinate of tiotropium.

Approx. 600 mg of the amorphous tiotropium succinate are dissolved in 4 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of 1-butanol was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to a final temperature of 5° C. At this temperature the plate remained for a hold time of 24 h. The plates are opened afterwards the solid is obtained by evaporation of the solvent at room temperature in a vacuum chamber (13 kPa). Melting point: 193±3° C. (DSC); table 22 summarizes the X-ray powder reflections obtained for this form.

TABLE 22

| 2Θ [°] | d [Å] | I/Iₒ [%] |
|---|---|---|
| 8.22 | 10.74 | 14 |
| 10.42 | 8.48 | 13 |
| 11.18 | 7.90 | 53 |
| 13.34 | 6.63 | 19 |
| 14.98 | 5.91 | 24 |
| 16.50 | 5.37 | 23 |
| 17.38 | 5.10 | 34 |
| 18.74 | 4.73 | 100 |
| 20.50 | 4.33 | 17 |
| 21.74 | 4.08 | 92 |
| 22.58 | 3.93 | 63 |
| 24.02 | 3.71 | 6 |
| 25.18 | 3.53 | 7 |
| 25.70 | 3.47 | 8 |
| 26.74 | 3.33 | 13 |
| 28.14 | 3.17 | 85 |
| 29.90 | 2.98 | 25 |

### Example 23

### Crystalline Anhydrous Tiotropium Malonate

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate to loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of malonic acid in 10 ml of water. The salt solution was concentrated to about 15-20 ml, frozen in a 100 ml jar at −20° C. and placed in the freeze dryer at a final pressure of <0.05mbar (over night). A white fluffy cake that could be loosened-up easily was the result.

Approx. 500 mg of the amorphous tiotropium malonate are dissolved in 5 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a ethylene glycol was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to 30° C. and than with cooling rate of 1° C./min to a final temperature of 5° C. At this temperature the plate remained for a hold time of 24 h. The plates are opened afterwards the solid is obtained by evaporation of the solvent at room temperature in a vacuum chamber (13 kPa). Table 23 summarizes the X-ray powder reflections obtained for this form.

TABLE 23

| 2Θ [°] | d [Å] | I/Iₒ [%] |
|---|---|---|
| 7.30 | 12.10 | 15 |
| 7.82 | 11.29 | 5 |
| 8.58 | 10.29 | 24 |
| 11.34 | 7.79 | 9 |
| 12.22 | 7.23 | 11 |
| 13.26 | 6.67 | 97 |
| 13.82 | 6.40 | 13 |
| 15.02 | 5.89 | 10 |
| 17.22 | 5.14 | 74 |
| 18.02 | 4.92 | 79 |
| 19.42 | 4.57 | 98 |
| 20.22 | 4.39 | 97 |
| 21.50 | 4.13 | 20 |
| 23.26 | 3.82 | 13 |
| 23.78 | 3.74 | 7 |
| 24.74 | 3.59 | 8 |
| 25.42 | 3.50 | 29 |
| 25.94 | 3.43 | 8 |
| 26.82 | 3.32 | 54 |
| 27.34 | 3.26 | 7 |
| 27.94 | 3.19 | 14 |
| 28.38 | 3.14 | 11 |
| 29.42 | 3.03 | 11 |

### Example 24

### Crystalline Tiotropium Malate N,N-Dimethylacetamide (DMA) Solvate (Form I)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of L-malic acid in 10 ml of water. The salt solution was concentrated to about 15-20 ml, frozen in a 100 ml jar at −20° C. and placed in the freeze dryer at a final pressure of <0.05mbar (over night). A white fluffy cake that could be loosened-up easily was the result.

Approx. 500 mg of the amorphous tiotropium malate are dissolved in 5 ml of a mixture of acetone/water=80:20. In two steps 75 μl (40+35 μl) of this stock solution is transferred to into one of the small vials of a 96 well plate. The plate containing the stock solution was placed in a vacuum chamber (1 kPa) at room temperature for 24 h for each dosing step. After the stock solvent was evaporated 30 μl of a N,N-dimethyl-acetamide was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to 30° C. and than with cooling rate of 1° C./min to a final temperature of 5° C. At this temperature the plate remained for a hold time of 24 h. The plates are opened afterwards the solid is obtained by evaporation of the solvent at room temperature in a vacuum chamber (13 kPa). Melting point: 170+5° C. (DSC; under decomposition); the crystal structure of this salt was solved by single crystal X-ray diffraction analysis (see table 24).

TABLE 24

| | |
|---|---|
| Empirical formula | C₁₉H₂₂NO₄S₂⁺•C₄H₅O₅⁻•C₄H₉NO |
| Fw | 612.70 |
| T [K] | 293(2) |
| λ [Å] | 0.71073 |
| Crystal system | Monoclinic |
| Space group | P2₁ |
| Unit cell dimensions | |
| a [Å] | 7.4670(5) |
| b [Å] | 14.4950(9) |
| c [Å] | 14.0490(14) |
| β | 100.095(2) |
| V [Å³] | 1497.0(2) |
| Z | 2 |
| Dₘ [g/cm³] | 1.359 |
| F(000) | 648 |
| Crystal size [mm³] | 0.3 × 0.25 × 0.2 |
| θ range [°] | 2.77 → 27.48 |
| Reflections collected | 4633 |
| Independent reflections | 3810 [Rᵢₙₜ = 0.0659] |
| Data/restraints/parameters | 3810/1/382 |
| S | 1.078 |
| R [I > 2σ(I)] | R1 = 0.0678, wR2 = 0.1441 |
| R indices (all data) | R1 = 0.1085, wR2 = 0.1657 |
| Absolute structure parameter | 0.14(17) |
| (Flack parameter) | |

### Example 25

### crystalline tiotropium malate 1-methyl-2-pyrollidinone (NMP) solvate (form II)

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of to 10 μm. The collector vial was previously charged with 1.1 equivalents of L-malic acid in 10 ml of water. The salt solution was concentrated to about 15-20 ml, frozen in a 100 ml jar at −20° C. and placed in the freeze dryer at a final pressure of <0.05mbar (over night). A white fluffy cake that could be loosened-up easily was the result.

Approx. 500 mg of the amorphous tiotropium malate are dissolved in 5 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a NMP/methanol=60:40 was added to this vial. The whole 96 well plate is sealed afterwards and heated up with a heating rate of 5° C./min to 50° C. at which the plate stays for an additional 30 minutes. Afterwards the plate is cooled with a cooling rate of 5° C./h to 30° C. and than with cooling rate of 1° C./min to a final temperature of 5° C. At this temperature the plate remained for a hold time of 24 h. The plates are opened afterwards the solid is obtained by evaporation of the solvent at room temperature in a vacuum chamber (13 kPa). Melting point: 170±5° C. (DSC; under decomposition); table 25 summarizes the X-ray powder reflections obtained for this form.

TABLE 25

| 2Θ [°] | d [Å] | I/Iₒ [%] |
|---|---|---|
| 6.46 | 13.67 | 26 |
| 8.86 | 9.97 | 53 |
| 12.18 | 7.26 | 25 |
| 13.66 | 6.47 | 100 |
| 14.14 | 6.26 | 38 |
| 14.94 | 5.92 | 84 |
| 15.70 | 5.69 | 22 |
| 16.14 | 5.49 | 36 |
| 16.86 | 5.25 | 54 |
| 17.30 | 5.12 | 68 |
| 17.62 | 5.09 | 53 |
| 19.30 | 4.59 | 40 |
| 20.06 | 4.49 | 35 |
| 21.46 | 4.14 | 23 |
| 22.42 | 3.96 | 43 |
| 22.90 | 3.88 | 27 |
| 23.74 | 3.74 | 25 |
| 24.50 | 3.63 | 42 |
| 25.34 | 3.51 | 64 |
| 25.78 | 3.45 | 41 |
| 26.74 | 3.33 | 15 |
| 27.62 | 3.23 | 16 |
| 28.82 | 3.09 | 15 |
| 29.42 | 3.03 | 8 |

### Example 26

### Crystalline Tiotropium Oxalate Dihydrate

1.00 g of tiotropium bromide monohydrate (according to WO 02/30928) was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of oxalic acid in 10 ml of water. The salt solution was concentrated to about 15-20 ml, frozen in a 100 ml jar at −20° C. and placed in the freeze dryer at a final pressure of <0.05mbar (over night). A white fluffy cake that could be loosened-up easily was the result.

Approx. 800 mg of the amorphous tiotropium oxalate are dissolved in 5 ml of a mixture of acetone/water=80:20. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a mixture of 1,2-dimethoxyethane/methanol 60:40 was added to this vial. The whole 96 well plate is sealed afterwards and heated up with a heating rate of 5° C./min to 50° C. at which the plate stays for an additional 30 minutes. Afterwards the plate is cooled with a cooling rate of 10° C./h to a final temperature of 5° C. At this temperature the plate remained for a hold time of 24 h. The plates are opened afterwards the solid is obtained by evaporation of the solvent at room temperature in a vacuum chamber (13 kPa).

The crystal structure of this salt was solved by single crystal X-ray diffraction analysis (see table 26).

TABLE 26

| | |
|---|---|
| Empirical formula | C₁₉H₂₂NO₄S₂⁺•C₂O₄H⁻•2H₂O |
| Fw | 517.56 |
| T [K] | 293(2) |
| λ [Å] | 0.71073 |
| Crystal system | Monoclinic |
| Space group | P 2₁/c |
| Unit cell dimensions | |
| a [Å] | 11.4540(4) |
| b [Å] | 10.0620(4) |
| c [Å] | 20.2480(9) |
| β [°] | 95.969(2) |
| V [Å3] | 2320.93(16) |
| Z | 4 |
| Dₘ [g/cm³] | 1.481 |
| F(000) | 1088 |
| Crystal size [mm3] | 0.3 × 0.3 × 0.1 |
| θ range [°] | 2 → 28.5°. |
| Reflections collected | 5184 |
| Independent reflections | 5184 [Rᵢₙₜ = 0.0000*] |
| Data/restraints/parameters | 5184/0/331 |
| S | 1.153 |
| R [I > 2σ(I)] | R1 = 0.1009, wR2 = 0.2111 |
| R indices (all data) | R1 = 0.1325, wR2 = 0.2315 |

### Example 27

### Crystalline Anhydrous Tiotropium p-Toluenesulphonate (Form II)

1.00 g of tiotropium bromide monohydrate was dissolved in 50 ml of demineralised water at 25° C. 10 ml of bicarbonate loaded IER slurry was added to this solution and stirred for 5 minutes before a second equivalent of 5 ml bicarbonate loaded IER slurry was added. The mixture was stirred for additional 5 minutes. A silver nitrate test as described above indicated the completion of the exchange. After stirring for an additional 5 minutes the slurry was filtered with PTFE filters with a pore size of 10 μm. The collector vial was previously charged with 1.1 equivalents of p-toluenesulfonic acid in 10 ml of water. From the resulting solution water was evaporated under vacuum over a water bath which was kept below 35° C. The obtained solid was further dried under vacuum yielding an amorphous oil of the tosylate of tiotropium. The amorphous oil was dissolved in acetonitril/water (70:30) and freeze dried over night. An amorphous solid material was obtained.

Approx. 480 mg of the amorphous tiotropium tosylate are dissolved in 10 ml of a mixture of acetonitrile/water=70:30. In two steps 75 μl (40+35 μl) of this stock solution is transferred into one of the small vials of a 96 well plate. The plate containing the stock solution was placed in a vacuum chamber (1 kPa) at room temperature for 24 h for each dosing step. After the stock solvent was evaporated 30 μl of a chloroform was added to this vial. The whole 96 well plate is sealed afterwards and heated up with a heating rate of 5° C./min to 50° C. at which the plate stays for an additional 30 minutes. Afterwards the plate is cooled with a cooling rate of 30° C./h to 3° C. At this temperature the plate remained for a hold time of 1 h. The plates are opened afterwards the solid is obtained by evaporation of the solvent at room temperature in a vacuum chamber (13 kPa). Melting point: 140±5° C. (DSC); table 27 summarizes the X-ray powder reflections obtained for this form.

TABLE 27

| 2Θ [°] | d [Å] | I/Iₒ [%] |
|---|---|---|
| 4.74 | 18.64 | 14 |
| 9.62 | 9.19 | 15 |
| 11.02 | 8.03 | 19 |
| 11.74 | 7.54 | 37 |
| 13.50 | 6.56 | 36 |
| 14.26 | 6.21 | 8 |
| 15.22 | 5.82 | 11 |
| 16.10 | 5.50 | 12 |
| 16.58 | 5.35 | 6 |
| 17.46 | 5.08 | 100 |
| 18.10 | 4.90 | 20 |
| 18.78 | 4.72 | 26 |
| 19.82 | 4.48 | 47 |
| 20.50 | 4.33 | 7 |
| 21.42 | 4.15 | 26 |
| 22.10 | 4.02 | 11 |
| 22.54 | 3.94 | 18 |
| 23.18 | 3.84 | 71 |
| 24.02 | 3.70 | 12 |
| 25.94 | 3.43 | 6 |
| 26.54 | 3.36 | 9 |
| 27.30 | 3.27 | 26 |
| 28.14 | 3.17 | 10 |
| 28.98 | 3.08 | 8 |
| 29.18 | 3.06 | 6 |
| 29.90 | 2.99 | 11 |

### Example 28

### Crystalline Tiotropium Methanesulphonate Monohydrate (Form II)

Approximately 60 mg of the crystalline tiotropium methanesulphonate (anhydrous form I disclosed in WO 05/042528) are suspended in 2 ml of diisobutyl ketone. The slurry was stirred for 30 minutes at 60° C. Afterwards the solvent was gently evaporated at room temperature in a vacuum chamber (13 kPa). The crystalline material obtained was tiotropium methanesulphonate, form II which is hydrated form (monohydrate) of tiotropium methanesulphonate. Melting point: 235±3° C. (DSC); table 28 summarizes the X-ray powder reflections obtained for this form.

TABLE 28

| 2Θ [°] | d [Å] | I/Iₒ [%] |
|---|---|---|
| 10.22 | 8.65 | 3 |
| 10.94 | 8.09 | 3 |
| 12.34 | 7.17 | 21 |
| 13.18 | 6.72 | 15 |
| 14.02 | 6.32 | 31 |
| 15.00 | 5.91 | 16 |
| 15.70 | 5.64 | 11 |
| 15.90 | 5.57 | 6 |
| 16.34 | 5.42 | 13 |
| 17.10 | 5.18 | 25 |
| 17.90 | 4.95 | 26 |
| 18.44 | 4.81 | 22 |
| 19.86 | 4.47 | 100 |
| 21.74 | 4.09 | 47 |
| 22.50 | 3.95 | 12 |
| 22.82 | 3.90 | 12 |
| 23.50 | 3.79 | 39 |
| 25.14 | 3.54 | 35 |
| 26.66 | 3.34 | 27 |
| 27.90 | 3.20 | 21 |
| 28.22 | 3.16 | 30 |
| 29.46 | 3.03 | 17 |
| 30.06 | 2.97 | 9 |
| 31.66 | 2.83 | 23 |
| 32.18 | 2.78 | 24 |

1-16. (canceled)

17. A compound of formula 2 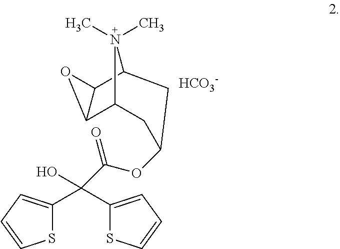
optionally in the form of solvates or hydrates thereof.

18-72. (canceled)
